(19) United States
(12) Patent Application Publication
(10) Pub. No.: US 2010/0151520 A1
(43) Pub. Date: Jun. 17, 2010

(54) Translation Enhancer Elements Of Genes Encoding Human Tau Protein and Human Alpha-Synuclein Protein

(76) Inventors: Jack Rogers, Arlington, MA (US); Avi Friedlich, Charlestown, MA (US)

Correspondence Address: WOLF GREENFIELD & SACKS, P.C., 600 ATLANTIC AVENUE, BOSTON, MA, 02210-2206, US

(21) Appl. No.: 12/638,494

(22) Filed: Dec. 15, 2009

Related U.S. Application Data
(62) Division of application No. 11/316,339, filed on Dec. 22, 2005, now abandoned.
(60) Provisional application No. 60/639,072, filed on Dec. 22, 2004.

(51) Int. Cl. C12P 21/02; C07H 21/00; C12N 15/74; C12N 5/10

(52) U.S. Cl. 435/69.1; 536/23.1; 435/320.1; 435/325

(57) ABSTRACT

The invention relates to translation enhancer elements that enhance translation of the gene encoding the human microtubule-associated tau protein and nucleic acid molecules that enhance translation of the gene encoding the human α-synuclein protein. The translation enhancer elements of the invention are useful in compositions and methods for identifying compounds for the prevention and/or treatment of neurodegenerative disease. The invention also includes in some aspects, vectors that include a translation enhancer element of the invention. The invention also includes the use of enhancer element containing vectors in methods to produce recombinant protein and in assays to identify compounds that modulate expression of tau protein or α-synuclein protein.

### RELATED APPLICATIONS

This application is a division of U.S. application Ser. No. 11/316,339, filed Dec. 22, 2005, which claims benefit under 35 U.S.C. §119 of U.S. provisional application Ser. No. 60/639,072, filed Dec. 22, 2004, the full contents of which are incorporated herein in their entirety.

### GOVERNMENT SUPPORT

The invention was made with government support under grant number R01AG21081 from the National Institutes of Health (NIH). The United States Government has certain rights in the invention.

### FIELD OF THE INVENTION

The invention relates in some aspects to translation enhancer elements: nucleic acid molecules that enhance translation of the gene encoding the human microtubule-associated tau protein and nucleic acid molecules that enhance translation of the gene encoding the human α-synuclein protein. The translation enhancer elements of the invention may be used in compositions and methods for identifying compounds for the prevention and/or treatment of neurodegenerative disease. The invention also includes in some aspects, vectors that include a translation enhancer element of the invention. The invention also includes the use of such vectors in methods to produce recombinant polypeptides and in assays to identify compounds that modulate expression of tau protein and/or α-synuclein protein.

### BACKGROUND OF THE INVENTION

Parkinson disease (PD) was first described by James Parkinson in 1817. It is a disease that affects over a half million Americans. Although most people affected by the disease are over 50 at the onset of the disease, there are also younger Parkinson's disease patients. Parkinson's disease is a neurodegenerative disease with clinical symptoms including tremor, muscular stiffness and difficulty with balance and walking. A classic pathological feature of the disease is the presence of an inclusion body, called the Lewy body, in many regions of the brain.

Environmental factors such as viral infections or neurotoxins were believed to be the most likely cause of Parkinson's disease. However, recent research indicates that in some instances Parkinson's disease may be a heritable disease. A candidate gene for some cases of Parkinson's disease has now been mapped to chromosome 4. Recessive mutations in the gene, which encodes α-synuclein protein, have been shown to cause early onset genetic forms of Parkinson's disease. (Papadimitriou, A., et al., 1999 Neurology 52:651-654). α-synuclein is a 15 kDa protein (monomer) that is ubiquitously expressed in all tissues, but aggregates of to α-synuclein cause a profound neurotoxicity in the neurons of the substantia nigra of Parkinson's Disease patients (Sulzer, D. 2001 Nat. Med. 7:1280-1282).

Alzheimer's disease (AD) is another neurodegenerative disease that is widespread in the population. AD is becoming increasingly common as the percentage of elderly in the population increases. Some patients present with symptoms of AD as early as age 30 or 40 and other patients do not exhibit symptoms until they reach their late 70 s or 80 s. Familial cases with a defined inheritance pattern account for only 5 to 10% of AD cases. Familial AD has been linked to gene defects on chromosomes 1, 12, 14, 19, and 21.

The clinical symptoms of AD include dementia and cognitive decline. The physiological manifestations at the cellular level include fibrillar aggregates of beta-amyloid that are toxic to neurons. Mutations in the tau gene, which codes for tau, a protein that is associated with microtubules, can be found in some AD cases. The abnormal tau may account for helical filaments found in neurofibrillary tangles. Tau-containing lesions have been identified in a number of brain disorders, and insoluble tau-containing tangles may build up and form one of the key pathological features of Alzheimer's disease (AD).

There has been a continuing effort to determine the basis for various neurodegenerative diseases include AD and PD. One mechanism that has been proposed as a possible factor in cell death in neurodegenerative disease is iron homeostasis. (Faucheux, B. A. & Hirsch, E. C. 1998 Biol Clin (Paris) 56 Spec No, 23-30). Intracellular iron homeostasis is regulated at the post transcriptional level by iron responsive elements (IREs), which are RNA stemloops that control ferritin translation, iron storage, and transferrin receptor mRNA (TfR mRNA for iron transport) for message stability (Thomson, A. M, et al., 1999 Int J Biochem Cell Biol 31:1139-1152). The ferritin IRE stemloop regulates translation of the light (L) and heavy (H) ferritin subunits in response to iron by a well-described pathway by which iron influx removes iron-regulatory protein-1 (IRP-1) and iron-regulatory protein-2 (IRP-2) from the 5′ cap specific IRE stemloops. Under conditions of iron chelation IRP-1 and IRP-2 serve as translational repressors. (Leedman, P., et al., 1996 J Biol Chem 271; 12017-12023). Although not clearly understood, it has been proposed that mechanisms involved in iron homeostasis may play a role in cell death in neurodegenerative disease.

Because of the severe impact of neurodegenerative disease on patients, and the increasing number of afflicted individuals in society, there is a clear need for methods treat these disorders. There is a lack of viable treatment options for the growing number of patients with neurodegenerative diseases such as PD and AD. Thus there is a urgent need for to the identification of therapeutic compounds that can be used for the prevention and/or treatment of neurodegenerative disease. The discovery of additional targets and tools with which to develop therapeutic compounds and regimens would advance the now-limited therapeutic options for these devastating diseases.

### SUMMARY OF THE INVENTION

We have identified translation enhancer elements of genes that encode tau protein and α-synuclein protein and have discovered that the enhancer elements are useful for the identification of compounds that modulate expression of tau protein or α-synuclein protein. The invention includes in some aspects assays to identify candidate agents that modulate tau protein or α-synuclein protein expression. Thus, the invention relates to compositions and assays to identify candidate agents that modulate tau protein or α-synuclein protein expression, and are therapeutically useful for the prevention and or treatment of Alzheimer's disease, Parkinson's disease, and other α-synuclein-associated and tau-associated diseases.

According to one aspect of the invention, isolated nucleic acid molecules are provided. The isolated nucleic acid molecules include an α-synuclein translation enhancer element that includes the nucleotide sequence set forth as SEQ ID NO:1 or a fragment or variant thereof and a polypeptide-encoding nucleic acid sequence operably linked to the translation enhancer element. In some embodiments, the nucleotide sequence of the α-synuclein enhancer element includes the sequence set forth as SEQ ID NO:1. In some embodiments, the fragment of SEQ ID NO:1 is SEQ ID NO:21. In some embodiments, the polypeptide-encoding nucleic acid sequence is a non-homologous polypeptide-encoding nucleic acid sequence. In certain embodiments, the 3′ nucleotide of the translation enhancer element is between 0 and about 100 nucleotides 5′ to the 5′ nucleotide of the polypeptide-encoding nucleic acid sequence. In some embodiments, the 3′ nucleotide of the translation enhancer element is between about 10 and 100 nucleotides 5′ to the 5′ nucleotide of the polypeptide-encoding nucleic acid sequence.

According to another aspect of the invention, vectors for expressing a recombinant polypeptide in a eukaryotic cell are provided. The vectors include a promoter that is active in the eukaryotic cell; an a synuclein translation enhancer element that includes the nucleotide sequence set forth as SEQ ID NO:1 or a fragment or variant thereof, wherein the enhancer element is 3′ to the promoter; and a nucleic acid sequence that encodes the recombinant polypeptide, wherein the nucleic acid sequence is 3′ to the translation enhancer element, and to is operably linked to the promoter. In some embodiments, the nucleotide sequence of the α-synuclein translation enhancer element includes the nucleotide sequence set forth as SEQ ID NO:1. In some embodiments, the fragment of SEQ ID NO:1 is SEQ ID NO:21. In certain embodiments, the nucleic acid sequence is non-homologous to the translation-enhancer element. In some embodiments, the 3′ nucleotide of the translation enhancer element is between 0 and about 100 nucleotides 5′ to the 5′ nucleotide of the nucleic acid sequence. In some embodiments, the 3′ nucleotide of the translation enhancer element is between about 10 and 100 nucleotides 5′ to the 5′ nucleotide of the nucleic acid sequence.

According to yet another aspect of the invention, isolated host cells transformed with a vector of any of the foregoing aspects and embodiments of the invention are provided.

According to another aspect of the invention, methods for producing a recombinant polypeptide are provided. The methods include growing host cells transformed with a vector of any of the foregoing aspects or embodiments of the invention, and purifying the recombinant polypeptide from either the host cells or the medium surrounding the host cells. In some embodiments, the last 3′ nucleotide of the translation enhancer element is between 0 and about 100 nucleotides 5′ to the first 5′ nucleotide of the nucleic acid sequence. In certain embodiments, the last 3′ nucleotide of the translation enhancer element is between about 10 and 100 nucleotides 5′ to the first 5′ nucleotide of the nucleic acid sequence.

According to another aspect of the invention, recombinant polypeptides are provided. The recombinant polypeptides are produced using any of the foregoing methods. In some embodiments, the production methods also include contacting the transformed host cells with an inducer in an amount sufficient to significantly increase polypeptide production. In some embodiments, the inducer is a cytokine. In some embodiments, the cytokine is interleukin-1α or interleukin-1β.

According to yet another aspect of the invention, methods of identifying a compound that modulates α-synuclein expression is provided. The methods include contacting a vector of any of the foregoing aspects of the invention or a host cell of any of the foregoing aspects of the invention with a test compound, determining a level of expression of the recombinant polypeptide in the absence and in the presence of the test compound, and comparing the level of expression of the recombinant polypeptide in the absence and in the presence of the test compound, wherein an increase or decrease in the level of expression of the recombinant polypeptide in the presence of the test compound compared to the level of expression of the recombinant polypeptide in the absence of the test compound identifies that the test to compound modulates α-synuclein expression. In certain embodiments, the test compound includes a nucleic acid sequence complementary to a portion of the α-synuclein translation enhancer element that includes the nucleotide sequence set forth as SEQ ID NO:1 or a fragment or variant thereof that is about 10 or more nucleotides in length. In some embodiments, the fragment of SEQ ID NO:1 is SEQ ID NO:21. In some embodiments, modulating α-synuclein expression is decreasing α-synuclein expression. In some embodiments, modulating α-synuclein expression is increasing α-synuclein expression.

According to yet another aspect of the invention, an isolated nucleic acid molecule is provided. The isolated nucleic acid includes a tau translation enhancer element that includes a nucleotide sequence set forth as SEQ ID NO:2, 3, 4, or 5 or a fragment or variant thereof and a polypeptide-encoding nucleic acid sequence operably linked to the translation enhancer element. In certain embodiments, the nucleotide sequence of the tau translation enhancer element includes a nucleotide sequence set forth as SEQ ID NOs: 2, 3, 4, or 5. In some embodiments, the polypeptide-encoding nucleic acid sequence is a non-homologous polypeptide-encoding nucleic acid sequence. In some embodiments, the first 5′ nucleotide of the translation enhancer element is between 0 and about 100 nucleotides 3′ to the last 3′ nucleotide of the polypeptide-encoding nucleic acid sequence. In some embodiments, the first 5′ nucleotide of the translation enhancer element is between about 10 and 100 nucleotides 3′ to the last 3′ nucleotide of the polypeptide-encoding nucleic acid sequence.

According to another aspect of the invention, vectors for expressing a recombinant polypeptide in a eukaryotic cell are provided. The vectors include a promoter that is active in the eukaryotic cell, a tau translation enhancer element that includes a nucleotide sequence set forth as SEQ ID NO:2, 3, 4, or 5 or a fragment or variant thereof, wherein the enhancer element is 3′ to the promoter; and a nucleic acid sequence that encodes the recombinant polypeptide, wherein the nucleic acid sequence is 5′ to the translation enhancer element, and is operably linked to the promoter. In certain embodiments, the nucleotide sequence of the tau translation enhancer element includes a nucleotide sequence set for as SEQ ID NOs: 2, 3, 4, or 5. In some embodiments, the nucleic acid sequence is non-homologous to the translation-enhancer element. In some embodiments, the first 5′ nucleotide of the translation enhancer element is between 0 and about 100 nucleotides 3′ to the last 3′ nucleotide of the polypeptide-encoding nucleic acid sequence. In certain embodiments, the first 5′ nucleotide of the translation enhancer element is between about 10 and 100 nucleotides 3′ to the last 3′ nucleotide of the polypeptide-encoding nucleic acid sequence.

According to yet another aspect of the invention, isolated host cells transformed with a vector of any of the foregoing aspects and embodiments of the invention are provided.

According to another aspect of the invention, methods for producing a recombinant polypeptide are provided. The methods include growing host cells transformed with a vector of any of the foregoing aspects or embodiments of the invention, and purifying the recombinant polypeptide from either the host cells or the medium surrounding the host cells. In some embodiments, the first 5′ nucleotide of the translation enhancer element is between 0 and about 100 nucleotides 5′ to the last 3′ nucleotide of the polypeptide-encoding nucleic acid sequence. In some embodiments, the first 5′ nucleotide of the translation enhancer element is between about 10 and 100 nucleotides 5′ to the last 3′ nucleotide of the polypeptide-encoding nucleic acid sequence.

According to another aspect of the invention, recombinant polypeptides are provided. The recombinant polypeptides are produced using any of the foregoing methods. In certain embodiments, the production methods also include contacting the transformed host cells with an inducer in an amount sufficient to significantly increase polypeptide production. In some embodiments, the inducer is a cytokine. In some embodiments, the cytokine is interleukin-1α or interleukin-1μ.

According to yet another aspect of the invention, methods of identifying a compound that modulates tau expression are provided. The methods include contacting a vector or host cell of any of the foregoing tau embodiments or aspects of the invention with a test compound, determining a level of expression of the recombinant polypeptide in the absence and in the presence of the test compound, and comparing the level of expression of the recombinant polypeptide in the absence and in the presence of the test compound, wherein an increase or decrease in the level of expression of the recombinant polypeptide in the presence of the test compound compared to the level of expression of the recombinant polypeptide in the absence of the test compound is an indication that the test compound modulates tau expression. In certain embodiments, the test compound includes a nucleic acid sequence complementary to a portion of a tau translation enhancer element that includes the nucleotide sequence set forth as SEQ ID NO:2, 3, 4, or 5 or a fragment or variant thereof that is about 10 or more nucleotides in length. In some embodiments, the sequence of the tau translation enhancer element includes a sequence set forth as SEQ ID NO:2, 3, 4, or 5. In some embodiments, modulating tau expression is decreasing tau expression. In some embodiments, modulating tau expression is increasing tau expression. These and other aspects of the invention will be described in further detail in connection with the detailed description of the invention.

### BRIEF DESCRIPTION OF THE FIGURES

FIG. 1 shows the sequence of a computer-generated predicted sequence of an α-synuclein (ASN) 5′ untranslated region stemloop compared with the iron responsive element (IRE) in the H-ferritin mRNA 5′ untranslated region. FIG. 1A shows the alignment of the 52 base α-synuclein 5′ untranslated region sequence (SEQ ID NO:1) and the +17 to +74 IRE subregion of the H-ferritin 5′ untranslated region (SEQ ID NO:6). FIG. 1B shows the region of core homology between the H-ferritin and α-synuclein 5′ untranslated regions, SEQ ID NOs:8 and 7 respectively). FIG. 1C-D show the predicted RNA folded structure of the α-synuclein 5′ untranslated region mRNA (SEQ ID NO:17) and H-ferritin mRNA (SEQ ID NO:18), respectively.

FIG. 2 shows an alternative α-synuclein (ASN) 5′UTR specific stemloop that was generated by manually pairing the bases that best fit a stem structure around the reference point CAGUGC in the α-synuclein mRNA 5′ untranslated region. FIG. 2A shows the alignment of the 52 base α-synuclein 5′ untranslated region sequence (SEQ ID NO:1) and the +17 to +74 IRE subregion of the H-ferritin 5′ untranslated region (SEQ ID NO:6). FIG. 2B shows the region of core homology between the H-ferritin and α-synuclein 5′ untranslated regions (SEQ ID NOs:8 and 7, respectively. FIG. 2C-D shows the predicted RNA folded structure of H-ferritin mRNA (SEQ ID NO:19) and the α-synuclein 5′ untranslated region mRNA (SEQ ID NO:20), respectively.

FIG. 3 is a digitized image of a western blot showing ferric ammonium citrate (FAC) induces an increase in α-synuclein protein levels. The blot shows that iron treatment of SY5Y neuroblastoma cells induced the steady-state levels of α-synuclein monomer (15 kDa). Lane 1 is untreated, Lane 2 is iron (FAC) treated for 6 hour and lane 3 is FAC treated for 3 days (100 μM FAC).

FIG. 4 is a histogram of quantitative real-time PCR data showing that α-synuclein mRNA levels are not modulated by iron chelation with desferioxamine (DFO) or iron influx with ferric ammonium citrate (FAC). Transferrin receptor mRNA (TfR mRNA) was the experimental positive control mRNA which was up-regulated by 3× fold in response to DFO and down-regulated 2× fold by FAC. Neuroblastoma (SY5Y) cells were treated with each agent for 6 hours or 16 hours and control cells were left untreated (U) for the same indicated times (i.e., 6 hours untreated=U6, and 16 hours untreated=U16).

FIG. 5 is a histogram of quantitative real-time PCR data showing that Tau mRNA levels are up-regulated by iron chelation with desferioxamine (DFO) and down regulated after iron influx with ferric ammonium citrate (FAC). Neuroblastoma cells (SY5Y) were treated with each agent for 6 hours or 16 hours and control cells were left untreated (U) for the same indicated times (i.e., 6 hours untreated=U6, and 16 hours untreated=U16). Transferrin receptor mRNA (TfR mRNA) was the experimental positive control mRNA which was up-regulated by 4.5× fold in response to DFO and 4-fold reduced by FAC.

FIG. 6 shows digitized images of gels showing a synuclein mRNA IP-RTPCR results from SH-SY5Y cells. FIG. 6A shows results showing the 1 kb ASN-specific PCR product that was an amplified cDNA derived from mRNA present in the supernatant and beads from immunoprecipitates of lysates prepared from untreated, iron-treated, and DFO-treated SH-SY5Y neuroblastoma cells. FIG. 6B shows the 1 kb ASN-specific PCR product that was an amplified cDNA derived from mRNA present in the supernatant and beads from immunoprecipitates of lysates prepared from untreated, iron-treated, and DFO-treated SH-SY5Y neuroblastoma cells. Tau mRNA did not selectively interact with either IRP-1 or IPR-2 whereas ASN mRNA was confirmed to interact with IPR-1 but not IPR-2 in SH-Sy5Y cells. (U=untreated, Fe=iron treated, DF=DFO treated).

FIG. 7 shows graphs indicating results of test of iron responsiveness and activation (Fe) and inhibition (DFO) of the luciferase reporter gene. The results indicate that α-synuclein 5′ UTR confers iron dependent activation and inhibition of a luciferase reporter gene. The α-synuclein 5′ UTR was found to confer Fe and DFO dependent translation of the luciferase reporter gene in a dose-dependent manner. FIG. 7A shows results from FAC treatment showing that with luciferase translation controlled by the α-synuclein 5′ UTR, treatment with 10 mg/ml FAC induced a 5 fold increase in luminescence, while treatment with 100 mg/ml induced a 20 fold increase (p<0.001). FIG. 7B shows results from DFO treatment showing that treatment with 50 mM DFO decreased luminescence by 25% and 100 mM DFO to decreased luminescence by 50% (p<0.001). FAC and DFO had no effect on luciferase activity with the wild-type pGL3 vector.

### DETAILED DESCRIPTION OF THE INVENTION

We have discovered that α-synuclein and tau protein each are involved in iron homeostasis. We have identified translation enhancer elements of genes that encode tau protein and α-synuclein protein and we have discovered that the enhancer elements are useful for the identification of compounds that modulate expression of tau protein or α-synuclein protein. The invention includes in some aspects assays to identify candidate agents that modulate tau protein or α-synuclein protein expression. Thus, the invention relates to compositions and assays to identify candidate agents that modulate tau protein or α-synuclein protein expression, and are therapeutically useful for the prevention and or treatment of Alzheimer's disease, Parkinson's disease, and other α-synuclein-associated and tau-associated diseases.

As used herein, the term “translation enhancer element” means a nucleotide sequence that can enhance the translation of an operably linked nucleic acid sequence. In some embodiments, the translation enhancer element is a tau translation enhancer element and can enhance translation of an operably linked nucleic acid sequence. In other embodiments of the invention, the translation enhancer element is an α-synuclein enhancer element and can enhance the translation of an operably linked nucleic acid sequence. As used herein, the term “operably linked” means genetic elements (e.g. nucleic acid sequences) that are joined in a manner that enables them to carry out their normal functions. For example, a nucleic acid sequence is operably linked to a promoter when its transcription is under the control of the promoter. In some embodiments, a nucleic acid sequence is operably linked to an α-synuclein or tau translation enhancer element. In certain embodiments the nucleic acid sequence that is operably linked to an α-synuclein or tau translation enhancer element is a non-homologous nucleic acid sequence. In some embodiments, a nucleic acid sequence that is operably linked to an α-synuclein or tau translational enhancer element as a reporter of activity of the α-synuclein or tau translation enhancer element, respectively, is a nucleic acid that encodes a detectable reporter such as luciferase reporter gene.

The α-synuclein enhancer elements of the invention include the nucleic acid sequence of α-synuclein 5′ untranslated region sequence set forth as SEQ ID NO:1. The α-synuclein enhancer elements of the invention also include fragments and variants of SEQ ID NO:1. Fragments and variants of SEQ ID NO:1 include nucleic acid sequences that have functionally insubstantial differences in sequence from SEQ ID NO:1, as evidenced by their retaining translation enhancing functional properties such as those of the α-synuclein translation enhancing element of SEQ ID NO:1. Minor substitutions, additions or deletions of nucleotides may take place within the sequence at positions without substantially affecting the enhancer element's ability to enhance the translation of an operably linked sequence. An α-synuclein enhancer element of the invention may also have a nucleotide sequence of an alternatively spliced α-synuclein 5′ untranslated region sequence.

The tau enhancer elements of the invention include nucleic acid sequences the same as that shown in SEQ ID NOs:2, 3, 4, and 5, as well as nucleic acid sequences with differences from SEQ ID NOs:2, 3, 4, and 5 that are functionally insubstantial, as evidenced by their retaining translation enhancing functional properties of the tau translation enhancer elements or SEQ ID NO:2, 3, 4, or 5. Minor substitutions, additions or deletions of nucleotides may take place within the sequence at positions without substantially affecting the enhancer element's ability to enhance the translation of an operably linked sequence.

Thus, the invention provides α-synuclein and tau translation enhancer elements set forth as SEQ ID NOs:1, 2, 3, 4, and 5 and fragments or variants thereof. A variant of one of SEQ ID NOs:1, 2, 3, 4, or 5 has a modified nucleic acid sequence of SEQ ID NO:1, 2, 3, 4, or 5, respectively. Such modifications may include additions, substitutions, and/or deletions of one or more nucleotides (e.g., from 1-20 nucleotides, including each integer in between) of the sequences provided as SEQ ID NOs:1-5. To be used in the methods and/or compositions of the invention, modified nucleic acid molecules substantially retain at least one activity or function of an unmodified nucleic acid molecule set forth as SEQ ID NO:1, 2, 3, 4, or 5, such as an ability to enhance translation.

It will be understood that variant nucleic acid molecules that are enhancer elements of the invention can be prepared and used in the methods and compositions of the invention. Thus, a translation enhancing element of the invention may be a nucleic acid that has a sequence set forth as SEQ ID NO:1, 2, 3, 4, or 5 with 1, 2, 3, 4, 5, 6, 7, 8, 9, 10, or 11 nucleotide substitutions. Additionally, an α-synuclein translation enhancer element may be shorter or longer than the sequence set forth as SEQ ID NO:1 as long as it substantially retains the function as an α-synuclein translation enhancer element. For example, an α-synuclein translation enhancer element of the invention may have a sequence set forth as SEQ ID NO:1 (or modified sequence thereof) that has 1, 2, 3, 4, 5, 6, 7, 8, 9, 10 nucleic acids to added and/or removed from one and/or both ends and be used in the methods and compositions of the invention. Similarly, a tau enhancer element of the invention may have a sequence set forth as SEQ ID NO:2, 3, 4, or 5 (or modified sequence thereof) that has 1, 2, 3, 4, 5, 6, 7, 8, 9, or 10 nucleic acids added and/or removed from one and/or both ends and be used in the methods and/or compositions of the invention.

An α-synuclein or tau translation enhancer element sequence that is shorter than one of SEQ ID NO:1, 2, 3, 4, or 5 is referred to herein as a fragment of SEQ ID NO:1, 2, 3, 4, or 5, respectively. For example, SEQ ID NO:21 is a fragment of SEQ ID NO:1 and can be used in the methods and products of the invention (see Example 4). Additional fragments of SEQ ID NO:1 that have α-synuclein translation enhancing function are also useful in aspects and embodiments of the invention as α-synuclein translation enhancing elements. Similarly, fragments of SEQ ID NOs:2, 3, 4, or 5 that have tau translation enhancing function are also useful in aspects of the invention as tau translation enhancing elements.

Numerous modified nucleic acid molecules that have one or more insertions, deletions, substitutions, etc. will be readily envisioned by one of skill in the art. Any of the foregoing nucleic acids can be tested by routine experimentation for retention of translation enhancing activity or structural relation to the nucleic acids disclosed herein.

As used herein, the term “non-homologous” means that the translation enhancer element is joined to a nucleic acid sequence that encodes a polypeptide other than the polypeptide that would be encoded by the nucleic acid sequence normally joined to the translation enhancer element in nature. For example, a nucleic acid that is non-homologous to an α-synuclein enhancer element of the invention would be a nucleic acid sequence that does not encode an α-synuclein polypeptide, and a nucleic acid that is non-homologous to a tau enhancer element of the invention would be a nucleic acid sequence that does not encode a tau polypeptide. A non-homologous nucleic acid sequence may serve as a marker for translation enhancing activity of an α-synuclein or tau translation enhancing element. For example, the effect on the expression of a non-homologous nucleic acid by the translation enhancing element reflects the effect on α-synuclein or tau expression by an α-synuclein or tau translation enhancing element, respectively. Thus, methods of the invention, in part, include the determination of the expression of such a non-homologous nucleic acid sequence as a indication of the activity of the translation enhancer element to which it is operably linked. As used herein, the term “promoter” means a region of DNA to which RNA polymerase binds before initiating the transcription of DNA into RNA. If the promoter is an to inducible promoter, its activity increases in response to an inducing agent.

As used herein, the term “complementary nucleotide sequence” refers to a sequence that would arise by normal base pairing. For example, the nucleotide sequence 5′-AGA-3′ would have the complementary sequence 5′-TCT′-3′. As used herein, the term “expression” is the process by which an mRNA is produced from DNA. The process of expression includes the transcription of the nucleic acid sequence into mRNA and in some instances, the subsequent translation of the mRNA into a polypeptide. As used herein, a nucleic acid sequence of the invention is a nucleic acid that is a template for a nucleic acid polymerase, in eukaryotes, RNA polymerase II. The nucleic acid molecules of the invention are transcribed into mRNAs that may then be translated into protein.

In some embodiments of the invention an α-synuclein or tau enhancer element of the invention may be inserted into a vector, e.g. an expression vector. A vector of the invention may be a cloning vector or an expression vector. A cloning vector is a DNA sequence (typically a plasmid or phage) that can replicate autonomously in a host cell, and which is characterized by one or a small number of unique restriction endonuclease recognition sites. A foreign nucleic acid molecule (DNA fragment) may be spliced into the vector at these sites in order to facilitate the replication and cloning of the fragment. The vector may also contain one or more markers suitable for use in the identification of cells containing the vector. For example, markers may provide antibiotic (e.g. tetracycline or ampicillin) resistance. An expression vector is similar to a cloning vector but is capable of inducing the expression of the nucleic acid molecule that has been cloned into it, after transformation into a host. The cloned nucleic acid molecule is usually placed under the control of (i.e., operably linked to) certain regulatory sequences such as promoters or enhancers. Promoter sequences may be constitutive, inducible, or repressible.

In some embodiments, the vector is in a host cell. As used herein, a host cell may be a prokaryotic or eukaryotic cell that is the recipient of a replicable expression vector or cloning vector. The term “host cell” encompasses a prokaryotic or eukaryotic cell that has been engineered to incorporate a desired nucleic acid sequence into its chromosome or in its genome. Examples of cells that can serve as hosts are well known in the art, as are techniques for cellular transformation (see e.g., Sambrook et al., Molecular Cloning: A Laboratory Manual, 2nd ed. Cold Spring Harbor (1989)).

The invention includes, in some aspects an isolated nucleic acid molecule that includes an α-synuclein translation enhancer element that includes the nucleotide sequence set to forth as SEQ ID NO: 1, and a polypeptide-encoding nucleic acid sequence that is operably linked to the translation enhancer element. In other aspects, the sequence of the α-synuclein translation enhancer may be a variant or fragment of the sequence of SEQ ID NO:1, as described herein. The polypeptide-encoding nucleic acid sequence can be a non-homologous polypeptide-encoding nucleic acid sequence. The methods of the invention, in part, include the determination of the expression of the nucleic acid sequence as a indication of the activity of the translation enhancer element to which it is operably linked. In some embodiments, the 3′ nucleotide of the α-synuclein translation enhancer element (e.g. SEQ ID NO:1 or variant or fragment thereof) is between 0 (e.g. adjacent to) and about 100 nucleotides 5′ to the 5′ nucleotide of the polypeptide-encoding nucleic acid sequence. An optimal distance can be routinely determined by one of ordinary skill in the art.

The invention also includes, in some aspects, vectors for expressing a recombinant polypeptide in a eukaryotic cell that include a promoter that is active in the eukaryotic cell, a translation enhancer element that includes the nucleotide sequence set forth as SEQ ID NO: 1, or variant or fragment thereof, wherein the enhancer is 3′ to the promoter; and a nucleic acid sequence that encodes the recombinant polypeptide, wherein the nucleic acid sequence is 3′ to the translation enhancer element, and is operably linked to the promoter. The nucleic acid sequence, which serves as an indicator of activity of the translation enhancer element, may be non-homologous to the translation-enhancer element. In some aspects of the invention, the 3′ nucleotide of the α-synuclein translation enhancer element (e.g. SEQ ID NO:1 or variant or fragment thereof) is between 0 (e.g. adjacent to) and about 100 nucleotides 5′ to the 5′ nucleotide of the polypeptide-encoding nucleic acid sequence. The optimal distance may be determined based on the specific vector used and can be routinely determined by one of ordinary skill in the art. In some aspects an isolated host cell is transformed with the vector. It will be understood that in some embodiments the α-synuclein translation enhancer has a sequence that is a modification sequence of SEQ ID NO:1 or variant or fragment thereof, as described herein.

The invention includes, in some aspects an isolated nucleic acid molecule that includes a Tau translation enhancer element that includes a nucleotide sequence set forth as SEQ ID NO: 2, 3, 4, or 5, or variant or fragment thereof, and a polypeptide-encoding nucleic acid sequence that is operably linked to the translation enhancer element. In other aspects, the tau translation enhancer may have a sequence that is a variant or fragment of a sequence of SEQ ID NO:2, 3, 4, or 5 as described herein. The polypeptide-encoding nucleic acid to sequence can be a non-homologous polypeptide-encoding nucleic acid sequence. The methods of the invention, in part, include the determination of the expression of the nucleic acid sequence as a indication of the activity of the translation enhancer element to which it is operably linked. In some aspects of the invention, the first 5′ nucleotide of the Tau translation enhancer element is between 0 (e.g. adjacent to) and about 100 nucleotides 3′ to the last 3′ nucleotide of the polypeptide-encoding nucleic acid sequence. The optimal distance can be routinely determined by one of ordinary skill in the art.

The invention also includes, in some aspects, vectors for expressing a recombinant polypeptide in a eukaryotic cell that include a promoter that is active in the eukaryotic cell, a translation enhancer element that includes the nucleotide sequence of SEQ ID NOs: 2, 3, 4, or 5, or variant or fragment thereof, wherein the enhancer is 3′ to the promoter; and a nucleic acid sequence that encodes the recombinant polypeptide, wherein the nucleic acid sequence is 5′ to the translation enhancer element, and is operably linked to the promoter. The nucleic acid sequence, which serves as an indicator of activity of the translation enhancer element, can be non-homologous to the translation-enhancer element. In some aspects of the invention, the first 5′ nucleotide of the translation enhancer element (e.g., SEQ ID NO:2, 3, 4, or 5, or variant or fragment thereof) is between 0 (e.g. adjacent to) and about 100 nucleotides 3′ to the last 3′ nucleotide of the polypeptide-encoding nucleic acid sequence. The optimal distance may be determined based on the specific vector used and can be routinely determined by one of ordinary skill in the art. In some aspects an isolated host cell is transformed with the vector. In other aspects, the tau translation enhancer may have a sequence that is a variant or fragment of the sequence of SEQ ID NO:2, 3, 4, or 5 as described herein.

A host cell that has been transformed with a vector that includes an α-synuclein translation enhancing element or a tau translation enhancing element as described herein, can be used in methods of the invention for producing a recombinant polypeptide. The methods include growing host cells transformed with the vector and purifying the recombinant polypeptide from either the host cells or the medium surrounding the host cells. Those of ordinary skill in the art will understand how to select and utilize vectors for these methods using only routine procedures. The methods of the invention can be used to produce a recombinant polypeptide. In some aspects of the invention, an inducer can be contacted with a transformed host cell (e.g. that include an α-synuclein or tau translation enhancer element) to increase the expression of a polypeptide encoded by a nucleic acid in the vector described above herein. Inducers are known in the art and include, for example, cytokines. Examples of cytokines that are useful in the methods of the invention include, but are not limited to interleukin-1α or interleukin-1β.

The invention also includes, in some aspects, use of a vector as described above, for identifying a compound that modulates α-synuclein or tau expression. These methods of the invention include contacting an α-synuclein vector or a tau vector (as described above) or a host cell that contains an α-synuclein or a tau vector, with a test compound. The level of expression of the recombinant polypeptide in the vector or cell can be determined following contact with the test compound and the level can be used as a measure of how the compound would affect activity of the α-synuclein or tau translation enhancing element and expression of α-synuclein or tau, respectively. The level of expression of the recombinant polypeptide following contact with the test compound can be compared to a control level of expression of the recombinant polypeptide. A control level may be the level of expression of the recombinant polypeptide prior to contact with the test compound and/or the level of an equivalent vector or host cell not contacted with the test compound. The level of expression of the recombinant polypeptide after contact can be compared with the control level of expression of the recombinant polypeptide and a statistically significant increase or decrease in the level of expression of the recombinant polypeptide in the presence of the test compound compared to the level of expression of the recombinant polypeptide in the control (e.g. in the absence of the test compound) identifies that the test compound modulates α-synuclein expression or tau expression.

As used herein with respect to nucleic acids and polypeptides, “isolated” means separated from its native environment and present in sufficient quantity to permit its identification or use. As used herein with respect to nucleic acids, the term “isolated” means: (i) amplified in vitro by, for example, polymerase chain reaction (PCR); (ii) recombinantly produced, e.g., by cloning; (iii) purified, for example, by cleavage and gel separation; or (iv) synthesized by, for example, chemical synthesis.

Isolated nucleic acids, proteins, or polypeptides may, but need not be, substantially pure. The term “substantially pure” means that the nucleic acid molecules or proteins or polypeptides are essentially free of other substances with which they may be found in nature or in vivo systems to an extent practical and appropriate for their intended use. Substantially pure nucleic acids and proteins may be produced by techniques well known in the art. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. An isolated nucleic acid as used herein is not a naturally occurring chromosome.

In some embodiments, “substantially pure” means that the nucleic acid or polypeptide of interest comprises at least 85% of a sample, with greater percentages preferred. Many methods of assessing the purity of nucleic acids and proteins within a sample are available, including, but not limited to: polyacrylamide gel electrophoresis, chromatography and analytical centrifugation. Because an isolated protein or nucleic acid molecule may be admixed with a pharmaceutically acceptable carrier in a pharmaceutical preparation, the protein or nucleic acid molecule may comprise only a small percentage by weight of the preparation. The protein or nucleic acid molecule is nonetheless isolated in that it has been separated from the substances with which it may be associated in living systems, e.g. isolated from other proteins or nucleic acids, respectively. As used herein the terms “protein” and “polypeptide” are used interchangeably.

It will also be recognized that the invention embraces the use of the α-synuclein and tau sequences in expression vectors, as well as to transfect host cells and cell lines, be these prokaryotic (e.g., E. coli), or eukaryotic (e.g., dendritic cells, neuronal cells, B cells, CHO cells, COS cells, yeast expression systems and recombinant baculovirus expression in insect cells). Especially useful are mammalian cells such as human, mouse, hamster, pig, goat, non-human primate, etc. The cells may be of a wide variety of tissue types, and include primary cells and cell lines. The expression vectors require that the pertinent sequence, i.e., those nucleic acids of the invention, be operably linked to a promoter. The functional copy of the nucleic acid sequence is under operable control of regulatory elements which permit expression of the nucleic acid sequence in the genetically engineered cell(s). Numerous transfection and transduction techniques as well as appropriate expression vectors are well known to those of ordinary skill in the art.

Various techniques may be employed for introducing nucleic acids of the invention to into cells, depending on whether the nucleic acids are introduced in vitro or in vivo in a host. Such techniques include transfection of nucleic acid-CaPO₄ precipitates, transfection of nucleic acids associated with DEAE, transfection or infection with the foregoing viruses including the nucleic acid of interest, liposome-mediated transfection, and the like. For certain uses, it is preferred to target the nucleic acid to particular cells. In such instances, a vehicle used for delivering a nucleic acid of the invention into a cell (e.g., a retrovirus, or other virus; a liposome) can have a targeting molecule attached thereto.

The invention includes assays to identify compounds that modulate the expression of α-synuclein or tau and/or to reduce the level of α-synuclein and/or tau in neurodegenerative disease, such as α-synuclein-associated disorders and/or tau-associated disorders. Thus the compositions and methods of the invention related to α-synuclein-associated disorders and tau-associated disorders. As used herein α-synuclein-associated disorders include, but are not limited to: synucleinopathies, Parkinson's disease, Lewy body disease, Alzheimer's disease. As used herein tau-associated disorders include, but are not limited to: Alzheimer's disease, Down's syndrome, frontal-temporal dementia, and progressive supranuclear palsy.

The assays described herein may be carried out in host cells and/or cells or samples obtained from subjects. As used herein, the host cell can be any prokaryotic or eukaryotic cell that is the recipient of a replicable expression vector or cloning vector. As used herein, a subject is a human, non-human primate, cow, horse, pig, sheep, goat, dog, cat, or rodent. In all embodiments, human subjects are preferred. The samples used herein may be any cell, body tissue, or body fluid sample obtained from a subject. In some embodiments, the cell or tissue sample includes neuronal cells and/or is a neuronal cell or tissue sample.

As described above, the invention relates in some aspects to the identification and testing of candidate α-synuclein or tau expression-modulating compounds. Candidate α-synuclein or tau translation-modulating compounds can be screened for modulating (enhancing or inhibiting) expression of α-synuclein or tau using the assays described herein (e.g., in the Example section). Using such assays, the expression-modulating compounds that have α-synuclein or tau expression enhancing or inhibiting activity can be identified. It is understood that any mechanism of action described herein for the α-synuclein or tau expression-modulating compounds is not intended to be limiting, and the scope of the invention is not bound by any such mechanistic descriptions provided herein.

The invention includes methods for screening for compounds that modulate the expression of α-synuclein protein and or tau protein. The methods of the invention include cell-based (in vitro and in vivo) assays of various kinds. Cell-based assays can include contacting a cell that has α-synuclein or tau expression with compounds that are candidate modulators of α-synuclein or tau translation. Cell-based assays may also include contacting a cell that has expression of a nucleic acid sequence that is in conjunction with an α-synuclein or tau translation enhancer element of the invention. In some embodiments, the nucleic acid sequence that is in conjunction with an α-synuclein or tau translation enhancer element is a non-homologous nucleic acid sequence.

The invention further provides methods of identifying pharmacological agents or lead compounds for agents and compounds that modulate α-synuclein or tau expression. Generally, the screening methods involve assaying for compounds that modulate (enhance or inhibit) the level of α-synuclein or tau expression, by assessing the effect of the compound on the expression of a nucleic acid sequence that is operably linked to the α-synuclein or tau translation enhancing element. In some embodiments, the nucleic acid sequence operably linked to an α-synuclein or tau translation enhancer element is a non-homologous nucleic acid sequence. As will be understood by one of ordinary skill in the art, the methods of the invention may be used to measure the level of α-synuclein or tau expression enhancement or reduction through the use of a nucleic acid sequence operably linked to an enhancer element of the invention, e.g., using a screening method described herein. In some embodiments, the nucleic acids sequence operably linked to an enhancer element of the invention is a non-homologous nucleic acid sequence.

A wide variety of assays for identifying pharmacological agents can be used in accordance with this aspect of the invention, including, expression assays utilizing α-synuclein or tau enhancer element activity of the invention. As used herein, the term “pharmacological agent” means an α-synuclein or tau expression-modulating compound. An example of such an assay that is useful to test candidate α-synuclein or tau expression-modulating compounds is provided in the Examples section. In such assays, the assay mixture comprises a candidate pharmacological agent. Typically, a plurality of assay mixtures is run in parallel with different agent concentrations to obtain a different response to the various concentrations. Typically, one of these concentrations serves as a negative control, i.e., at zero concentration of agent or at a concentration of agent below the limits of assay detection.

Some aspects of the invention include cell-based assays in which cells that have α-synuclein or tau enhancer element activity may be contacted with compounds that are candidate modulators of α-synuclein or tau expression. For the assays of the invention, compounds that modulate (either inhibit or enhance) α-synuclein or tau expression may be identified by determining the level of expression of a nucleic acid sequence operably linked to an α-synuclein or tau translation enhancer element, relative to a control cell or cell extract (e.g., a control that is not contacted with a candidate compound). In some embodiments, the nucleic acid sequence operably linked to an α-synuclein or tau translation enhancer element is a non-homologous nucleic acid sequence.

The invention includes the use of the aforementioned nucleic acid molecules in assays to identify compounds that modulate levels of α-synuclein or tau expression. The assays of the invention include, but are not limited to assays of the type described in the Examples, and include in vitro expression assessment assays, Western blot assays, and immunoassays, etc. The assay mixture comprises a candidate pharmacological agent(s), e.g. a candidate α-synuclein or tau expression modulator. The various assays used to determine the levels of expression of a nucleic acid sequence operably linked to an α-synuclein or tau enhancer element of the invention include the use of materials that specifically bind to an expression product of the nucleic acid sequence and materials that specifically bind to the nucleic acid sequence, gel electrophoresis; and the like. In some embodiments, the nucleic acid sequence operably linked to an α-synuclein or tau translation enhancer element is a non-homologous nucleic acid sequence.

Immunoassays may be used according to the invention including sandwich-type assays, competitive binding assays, one-step direct tests and two-step tests such as routinely practiced by those of ordinary skill in the art. It is contemplated that cell-based assays as described herein can be performed using cell samples and/or cultured cells. Cells of the invention include cells treated using methods described herein to modulate (e.g. inhibit or enhance) the level of expression of a nucleic acid sequence operably linked to an α-synuclein or tau enhancer element. In some embodiments, the nucleic acid sequence operably linked to an α-synuclein or tau translation enhancer element is a non-homologous nucleic acid sequence.

The candidate α-synuclein or tau expression-modulating molecules used in the assays of the invention can be natural or synthetic compounds, such as those in small molecule libraries of compounds (including compounds derived by combinatorial chemistry). Natural product libraries also can be screened using such methods, as can selected libraries of compounds known to exert pharmacological effects, such as libraries of FDA-approved to drugs. Compounds identified by the assays can be used in therapeutic methods of the invention described below.

As used herein, an “α-synuclein or tau expression-modulating compound” preferably is an α-synuclein or tau expression-inhibiting compound. An α-synuclein or tau expression-inhibiting compound is a specific molecule or combination of molecules, that inhibit α-synuclein or tau expression. Compounds that inhibit α-synuclein or tau expression include: small molecule compounds which typically are organic chemical compounds, RNAi and/or siRNA oligonucleotides that bind to α-synuclein-encoding or tau-encoding nucleic acid molecules according to complementary sequences. Such compounds preferably will reduce α-synuclein or tau expression by at least about 1.0%, 2.0%, 3.0%, 4.0%, 5.0%, 7.0%, 10%, 15%, 20%, 25%, 30%, 40%, 50%, or more. These and other compounds may be administered alone or in combination as part of a pharmaceutical composition.

Candidate α-synuclein or tau expression-modulating molecules of the invention encompass numerous chemical classes, although typically they are organic compounds. In some embodiments, the candidate pharmacological agents are small organic compounds, i.e., those having a molecular weight of more than 50 yet less than about 2500, preferably less than about 1000 and, more preferably, less than about 500. Candidate agents comprise functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

Candidate α-synuclein or tau expression-modulating molecules of the invention are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, to fungal, plant and animal extracts are available or readily produced. Additionally, natural and synthetically produced libraries and compounds can be readily be modified through conventional chemical, physical, and biochemical means. Further, known pharmacological agents can be tested and further may be subjected to directed or random chemical modifications such as acylation, alkylation, esterification, amidification, etc. to produce structural analogs of the agents.

The α-synuclein or tau expression-modulating molecules of the invention may include small molecules, polypeptides, (for example, competitive ligands and antibodies, or antigen-binding fragments thereof), and nucleic acids. For example, an α-synuclein or tau expression-modulating molecule may be a molecule that reduces translation of α-synuclein or tau, including nucleic acids that bind to other nucleic acids, [e.g. for antisense, RNAi, or small interfering RNA (siRNA) methods]. The methods of the invention also include, in some aspects, the administration of compounds that modulate, (e.g. reduce) expression of α-synuclein or tau polypeptides. For example, the methods of the invention may include the administration of molecules that are antisense of the nucleic acids that encode α-synuclein or tau translation enhancing element of the invention. The methods of the invention include in some embodiments, the use of RNAi and/or siRNA to inhibit α-synuclein or tau expression and activity.

As used herein, a “siRNA molecule” is a double-stranded RNA molecule (dsRNA) consisting of a sense and an antisense strand, which are complementary (Tuschl, T. et al., 1999, Genes & Dev., 13:3191-3197; Elbashir, S. M. et al., 2001, EMBO J., 20:6877-6888). In one embodiment the last nucleotide at the 3′ end of the antisense strand may be any nucleotide and is not required to be complementary to the region of the target gene. The siRNA molecule may be 19-23 nucleotides in length in some embodiments. In other embodiments, the siRNA is longer but forms a hairpin structure of 19-23 nucleotides in length. In still other embodiments, the siRNA is formed in the cell by digestion of double-stranded RNA molecule that is longer than 19-23 nucleotides. The siRNA molecule preferably includes an overhang on one or both ends, preferably a 3′ overhang, and more preferably a two nucleotide 3′ overhang on the sense strand. In another preferred embodiment, the two nucleotide overhang is thymidine-thymidine (TT). The siRNA molecule corresponds to at least a portion of a target sequence. In one embodiment the siRNA molecule corresponds to a region selected from a cDNA target sequence beginning between 50 to 100 nucleotides downstream of the start codon. In a preferred embodiment the to first nucleotide of the siRNA molecule is a purine. Many variations of siRNA and other double-stranded RNA molecules useful for RNAi inhibition of sequence expression will be known to one of ordinary skill in the art. In some embodiments, the siRNA molecules can be plasmid based.

In one aspect of the invention a vector comprising any of the nucleotide sequences of the invention is provided, preferably one that includes promoters active in mammalian cells. Non-limiting examples of vectors are the pSUPER RNAi series of vectors (Brummelkamp, T. R. et al., 2002, Science, 296:550-553; available commercially from OligoEngine, Inc., Seattle, Wash.). In one embodiment a partially self-complementary nucleotide coding sequence can be inserted into the mammalian vector using restriction sites, creating a stem-loop structure. In a preferred embodiment, the mammalian vector comprises the polymerase-III H1-RNA gene promoter. The polymerase-III H1-RNA promoter produces a RNA transcript lacking a polyadenosine tail and has a well-defined start of transcription and a termination signal consisting of five thymidines (T5) in a row. The cleavage of the transcript at the termination site occurs after the second uridine and yields a transcript resembling the ends of synthetic siRNAs containing two 3′ overhanging T or U nucleotides. Other promoters useful in siRNA vectors will be known to one of ordinary skill in the art.

Vector systems for siRNA expression in mammalian cells include pSUPER RNAi system described above. Other examples include but are not limited to pSUPER.neo, pSUPER.neo+gfp and pSUPER.puro (OligoEngine, Inc.); BLOCK-iT T7-TOPO linker, pcDNA1.2/V5-GW/lacZ, pENTR/U6, pLenti6-GW/U6-laminshrna and pLenti6/BLOCK-iT-DEST (Invitrogen, Carlsbad, Calif.). These vectors and others are available from commercial suppliers.

One set of embodiments of the invention includes the use of antisense molecules or nucleic acid molecules that reduce expression of genes via RNA interference (RNAi or siRNA). One example of the use of antisense, RNAi or siRNA in the methods of the invention is their use to decrease the level of α-synuclein or tau expression. The antisense oligonucleotides, RNAi, or siRNA nucleic acid molecules used for this purpose may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art-recognized methods, which may be carried out manually or by an automated synthesizer. They also may be produced recombinantly by vectors, e.g., as described above.

In some embodiments of the invention, the antisense or siRNA oligonucleotides also may include “modified” oligonucleotides. That is, the oligonucleotides may be modified in a number of ways, which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars that are covalently attached to low molecular weight organic groups other than a hydroxyl group at the 3′ position and other than a phosphate group at the 5′ position. Thus, modified oligonucleotides may include a 21-O-alkylated ribose group. In addition, modified oligonucleotides may include sugars such as arabinose instead of ribose.

A variety of other reagents also can be included in the assay mixtures of the invention. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

The assays of the invention may be used to identify candidate agents that modulate the expression of α-synuclein or tau. As described herein, the effect of a candidate agent on α-synuclein or tau expression may be determined indirectly using the methods of the invention, by the assessment of the level of expression of a non-homologous nucleic acid operably linked to an α-synuclein or tau translation enhancing element of the invention. In other embodiments, the effect of a candidate agent on α-synuclein or tau expression may be to determined directly using the methods of the invention, by the assessment of the level of expression of an α-synuclein polypeptide-encoding nucleic acid or tau-polypeptide-encoding nucleic acid. As used herein, the term “modulate” means to change, which in some embodiments means to “enhance” or “increase” and in other embodiments, means to “inhibit” or “reduce”. For example, in some embodiments, α-synuclein or tau expression is inhibited or reduced, and in some embodiments, α-synuclein or tau expression is enhanced or increased. It will be understood that reduction may mean reduction to zero or may mean reduction to a level below a normal level, a previous level, or a control level.

In general, the mixture of the foregoing assay materials is incubated under conditions whereby, but for the presence of the candidate pharmacological agent, a control level of expression of a nucleic acid operably linked to an α-synuclein or tau translation enhancing element of the invention will occur. In some embodiments, the nucleic acid sequence operably linked to an α-synuclein or tau translation enhancer element is a non-homologous nucleic acid sequence. It will be understood that a candidate pharmacological agent that is identified as a modulating agent may be identified as decreasing or eliminating α-synuclein or tau expression, as evidenced by the elimination or decrease of expression of a nucleic acid operably linked to an α-synuclein or tau translation enhancing element of the invention. A reduction of the expression of the nucleic acid operably linked to an α-synuclein or tau translation enhancing element need not be the absence of expression of the sequence, but may be a lower level of expression of the sequence than in a control.

The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 1 minute and 10 hours.

Levels of expression of a nucleic acid operably linked to an α-synuclein or tau translation enhancing element of the invention are preferentially compared to controls. The control may be a predetermined value, which can take a variety of forms. It can be a single value, such as a median or mean. It can be established based upon comparative groups (e.g. comparative cell types), such as in cells having normal amounts of expression of nucleic acid operably linked to an α-synuclein or tau translation enhancing element of the invention. It will be understood that the “normal” amount of expression of a nucleic acid sequence, will be to the amount that would result from the preparation of a nucleic acid operably linked to an α-synuclein or tau translation enhancing element of the invention in parallel with a similar construct, wherein one and not the other construct is contacted with the candidate modulatory compound. In some embodiments, a control may be the level of expression of a nucleic acid sequence from a construct that lacks the enhancer sequence. A control may also be the level of expression of a nucleic acids sequence from a construct that includes a mutated enhancer sequence. In some embodiments and controls, the nucleic acid sequence operably linked to an α-synuclein or tau translation enhancer element is a non-homologous nucleic acid sequence.

Once an compound or agent that modulates expression of a nucleic acid operably linked to an α-synuclein or tau translation enhancing element of the invention, has been identified, additional testing can be done to determine the effect of the compound on expression of α-synuclein or tau in cells and tissues. Cells for this secondary testing may be cells from subjects known to have a particular disease (e.g., Alzheimer's disease, Parkinson's disease, Down's syndrome, frontal-temporal dementia, progressive supranuclear palsy, Lewy body disease and synucleinopathies), condition or symptoms, and cells from groups without the disease, condition or symptoms. Another comparative cell type would be cells from subjects with a family history of a disease or condition and a group without such a family history.

The predetermined value of course, will depend upon the particular population of cells selected. For example, an apparently healthy cell population will have a different ‘normal’ range of α-synuclein or tau expression than will a population that is known to have a condition related to α-synuclein or tau expression. Accordingly, the predetermined value selected may take into account the category in which a cell type falls. Appropriate ranges and categories can be selected with no more than routine experimentation by those of ordinary skill in the art. By abnormal levels it is meant abnormal (high or low) relative to a selected control. Typically the control for the secondary level of testing will be based on apparently healthy normal cell types.

It will also be understood that the controls for use in the invention (e.g., in the primary or secondary assays of compounds) may be, in addition to predetermined values, samples of materials tested in parallel with the experimental materials. Examples include samples from control cells or control samples (e.g., generated through manufacture) to be tested in parallel with the experimental samples.

As mentioned above, it is possible to determine the efficacy of a candidate compound to modulate expression of a nucleic acid operably linked to an α-synuclein or tau translation enhancing element of the invention by monitoring changes in the absolute or relative amounts of the product encoded by the nucleic acid in the absence and/or presence of a candidate compound. For example, a decrease in the amount of the polypeptide encoded by the nucleic acid indicates that a candidate compound would decrease α-synuclein or tau expression. Similarly, an increase in the amount of the polypeptide encoded by the nucleic acid indicates that a candidate compound would increase α-synuclein or tau expression.

The ratio of expression of the nucleic acid operably linked to an α-synuclein or tau translation enhancing element of the invention, when contacted with the candidate compound, to the level of expression of the nucleic acid sequence operably linked to an α-synuclein or tau translation enhancing element, when not contacted with the candidate compound, provides an indication of the efficacy of a candidate compound's reduction of α-synuclein or tau expression, respectively. Accordingly, one can monitor levels of expression of a nucleic acid operably linked to an α-synuclein or tau translation enhancing element of the invention to determine the efficacy of a candidate compound for reduction of expression of α-synuclein or tau, respectively. Thus, using the assays of the invention, one can identify compounds for use in the prevention and/or treatment of conditions associated with abnormal α-synuclein or tau expression, which include, as described above, α-synuclein-associated and tau-associated disorders. In some embodiments of the invention, the nucleic acid sequence operably linked to an α-synuclein or tau translation enhancer element is a non-homologous nucleic acid sequence.

Changes in relative or absolute levels of expression of a nucleic acid operably linked to an α-synuclein or tau translation enhancing element of the invention greater than 0.1% when contacted with a candidate compound in an assay of the invention may indicate a compound that is effective for the prevention and/or treatment of a condition associated with abnormal α-synuclein or tau expression. As will be understood by those of ordinary skill in the art, a change in the level of expression that is a decrease indicates a reduction in the amount of α-synuclein or tau expression and a change that is an increase indicates an increase in the amount of α-synuclein or tau expression. Preferably, the change in levels of expression of a nucleic acid operably linked to an α-synuclein or tau translation enhancing element of the invention, which indicates a compound is effective, is greater than 0.2%, greater than 0.5%, greater than 1.0%, 2.0%, 3.0%, 4.0%, 5.0%, 7.0%, 10%, 15%, 20%, 25%, 30%, 40%, 50%, or to more. As described above, a decrease in expression of a nucleic acid operably linked to an α-synuclein or tau translation enhancing element of the invention in an assay of the invention, indicates a compound that may be useful to prevent and/or treat an α-synuclein-associated condition or a tau-associated condition.

It will be understood that a candidate pharmacological agent that is identified as a modulating agent may be identified as increasing or enhancing α-synuclein or tau expression, as evidenced by the increase or enhancement of expression of a nucleic acid operably linked to an α-synuclein or tau translation enhancing element of the invention. An increase in the sequence expression (and by extension, an increase in α-synuclein or tau expression) may be any significant increase from the level of expression in a control. An increase in expression of a nucleic acid operably linked to an α-synuclein or tau translation enhancing element may mean an increase from zero expression of the nucleic acid or may be an increase from a control level of expression of the nucleic acid to a higher level of expression of the nucleic acid. It will be understood that candidate agents and methods to increase the expression α-synuclein or tau proteins may be useful for research purposes, e.g. for making cell and/or animal models of disease conditions.

The assays described herein (see, e.g., the Examples section) include measuring the level of expression of a nucleic acid operably linked to an α-synuclein or tau translation enhancing element of the invention. Levels of expression of the nucleic acid operably linked to an α-synuclein or tau translation enhancing element of the invention be can be measured in a number of ways when carrying out the various methods of the invention. In one type of measurement, the level of expression of the nucleic acid is a measure of the level of the polypeptide encoded by the nucleic acid.

After incubation, the level of expression of the nucleic acid operably linked to an α-synuclein or tau translation enhancing element of the invention may be detected by any convenient method available to the user. One method of detection that is useful in the methods of the invention is the use of polypeptides, (e.g. antibodies), that specifically bind to the polypeptide encoded by the nucleic acid, or to specific fragments thereof. Detection may be effected in any convenient way for the assays of the invention. For example, an antibody may be coupled to a detectable label. For cell-based assays, one of the assay components may comprise, or be coupled to, a detectable label. A wide variety of detectable labels can be used, such as those that provide direct detection (e.g., radioactivity, a fluorophore, [e.g. Green Fluorescent Protein (GFP), Red Fluorescent Protein (RFP), etc.], a chromophore, Optical or electron density, etc.) or indirect detection (e.g., epitope tag such as the FLAG epitope, enzyme tag such as horseradish peroxidase, etc.).

A variety of methods may be used to detect the label, depending on the nature of the label and other assay components. Labels may be directly detected through optical or electron density, radioactive emissions, nonradiative energy transfers, etc. or indirectly detected with antibody conjugates, strepavidin-biotin conjugates, etc. Methods for detecting the labels are well known in the art.

The invention includes the use of agents (e.g., antibodies or antigen-binding fragments thereof) to determine the level of expression of a nucleic acid operably linked to an α-synuclein or tau translation enhancing element of the invention in the assays of the invention. As used herein, the term “antibodies” includes antibodies or antigen-binding fragments thereof. Antibodies of the invention can be identified and prepared that bind specifically to the expression product (or fragment thereof) of the nucleic acid operably linked to an α-synuclein or tau translation enhancing element of the invention

As used herein, “binding specifically to” means capable of distinguishing the identified material from other materials sufficient for the purpose to which the invention relates. Thus, “binding specifically to” an expression product of a nucleic acid operably linked to an α-synuclein or tau translation enhancing element of the invention means the ability to bind to and distinguish these molecules from other proteins. The antibodies and antigen-binding fragments thereof of the invention can be used for the assay of an expression product of a nucleic acid operably linked to an α-synuclein or tau translation enhancing element of the invention using known methods including, but not limited to enzyme linked immunosorbent (ELISA) assays, immunoprecipitations, and Western blots.

The antibodies of the present invention may be prepared by any of a variety of methods, including administering protein, fragments of protein, cells expressing the protein or fragments thereof and the like to an animal to induce polyclonal antibodies. The production of monoclonal antibodies is according to techniques well known in the art. As detailed herein, such antibodies or antigen-binding fragments thereof may be used for example to identify the presence of specific fragments the expression product of a nucleic acid operably linked to an α-synuclein or tau translation enhancing element of the invention as an indication of the efficacy of a candidate compound for reducing expression of α-synuclein or tau polypeptide. The antibodies of the invention include monoclonal and polyclonal antibodies.

Antibodies also may be coupled to specific labeling agents, for example, for imaging of cells and tissues with according to standard coupling procedures. Labeling agents include, but are not limited to, fluorophores, chromophores, enzymatic labels, radioactive labels, etc. Other labeling agents useful in the invention will be apparent to one of ordinary skill in the art.

Thus, antibodies and/or antigen-binding fragments thereof are useful in methods of the invention. With respect to the antibodies and antigen-binding fragments thereof, as is well known in the art, only a small portion of an antibody molecule, the paratope, is involved in the binding of the antibody to its epitope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab')₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd Fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Thus, for example, PCT International Publication Number WO 92/04381 teaches the production and use of murine RSV antibodies in which at least a portion of the murine FR regions have been replaced by FR regions of human origin. Such antibodies, including fragments of intact antibodies with antigen-binding ability, are often referred to as “chimeric” antibodies. Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab')₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab')₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or nonhuman sequences. The present invention also includes so-called single chain antibodies.

Thus, the invention involves polypeptides of numerous size and type that bind specifically to an expression product of a nucleic acid in association with an α-synuclein or tau translation enhancing element of the invention. In some embodiments, polypeptides that bind specifically to α-synuclein or tau polypeptide are also useful to determine the efficacy of a candidate compound to modulate (e.g., reduce) expression of α-synuclein or tau. The polypeptides may be derived also from sources other than antibody technology. For example, such polypeptide-binding agents can be provided by degenerate peptide libraries, which can to be readily prepared in solution, in immobilized form or as phage display libraries. Combinatorial libraries also can be synthesized of peptides containing one or more amino acids. Libraries further can be synthesized of peptoids and non-peptide synthetic moieties.

Some aspects of the invention include kits for assaying for compounds that modulate α-synuclein or tau expression and concomitant polypeptide production. Some aspects of the invention include kits for assaying for compounds that modulate α-synuclein or tau expression. Kits of the invention may include an α-synuclein or tau translation enhancing element of the invention and may include a nucleic acid sequence for use in the assay. In some embodiments, the nucleic acid sequence is a nucleic acid sequence that is non-homologous to the α-synuclein or tau enhancing element. Kits of the invention may also include vectors, promoters, control solutions or molecules for use in the assays of the invention.

Kits of the invention may also include molecules that bind to expression products, or fragments there of, of the nucleic acid molecule that is operably linked to an α-synuclein or tau translation enhancing element of the invention. The binding molecules may be antibodies or antigenic-fragments thereof and may be detectably labeled. As described herein, the binding molecules may be monoclonal or polyclonal antibodies that specifically bind to the expression product, or fragment thereof. The kit may also include materials for processing using procedures well known to those of skill in the art, to assess the level of expression of the nucleic acid molecule (e.g. a non-homologous nucleic acid molecule) as described herein. For example, procedures may include, but are not limited to, contact with a secondary antibody, or other method that indicates the presence of specific binding. The foregoing kits may also include instructions or other printed material on how to use the various components of the kits for identifying compounds that modulate expression of α-synuclein or tau polypeptide.

The methods of the invention can be used to screen or identify various compounds that are useful to decrease α-synuclein or tau polypeptide production. α-synuclein or tau polypeptide production may be decreased, e.g., for prevention and/or treatment of Alzheimer's disease, Parkinson's disease, Down's syndrome, frontal-temporal dementia, progressive supranuclear palsy, Lewy body disease or synucleinopathies, using methods to decrease the level of α-synuclein or tau polypeptide production.

The invention includes in some aspects the use of compounds that modulate α-synuclein or tau expression, which are identified using the assays of the invention, in therapeutic methods for the prevention and treatment of conditions associated with abnormal α-synuclein or tau polypeptide production. These methods of the invention include administration of α-synuclein or tau expression-modulating compounds, to decrease the level of α-synuclein or tau polypeptide production in cells or tissues. In general, the treatment methods of the invention involve administering an agent to modulate the level of α-synuclein or tau polypeptide production.

In certain embodiments, the method for treating a subject with a disorder characterized by abnormal levels of α-synuclein or tau polypeptide production involves administering to the subject an effective amount of a candidate compound identified through a method or assay of the invention. Various techniques may be employed for introducing α-synuclein or tau polypeptide expression-modulating compounds of the invention to cells or tissues, depending on whether the compounds are introduced in vitro or in vivo in a host. In some embodiments, the α-synuclein or tau expression-modulating compounds target neuronal cells and/or tissues. Thus, the α-synuclein or tau expression-modulating compounds can be specifically targeted to neuronal tissue (e.g. neuronal cells) using various delivery methods, including, but not limited to: administration to neuronal tissue, the addition of targeting molecules to direct the compounds of the invention to neuronal cells and/or tissues. Additional methods to specifically target molecules and compositions of the invention to brain tissue and/or neuronal tissues are known to those of ordinary skill in the art.

In some embodiments of the invention, an α-synuclein or tau expression-modulating compound of the invention may be delivered in the form of a delivery complex. The delivery complex may deliver the α-synuclein or tau expression-modulating compound into any cell type, or may be associated with a molecule for targeting a specific cell type. Examples of delivery complexes include an α-synuclein or tau-modulating compound of the invention associated with: a sterol (e.g., cholesterol), a lipid (e.g., a cationic lipid, virosome or liposome), or a target cell specific binding agent (e.g., an antibody, including but not limited to monoclonal antibodies, or a ligand recognized by target cell specific receptor). Some delivery complexes may be sufficiently stable in vivo to prevent significant uncoupling prior to internalization by the target cell. However, the delivery complex can be cleavable under appropriate conditions within the cell so that the α-synuclein or tau-modulating compound is released in a functional form.

An example of a targeting method, although not intended to be limiting, is the use of liposomes to deliver an α-synuclein or tau expression-modulating compound of the invention to into a cell. Liposomes may be targeted to a particular tissue, such as neuronal cells, by coupling the liposome to a specific ligand such as a monoclonal antibody, sugar, glycolipid, or protein. Such proteins include proteins or fragments thereof specific for a particular cell type, antibodies for proteins that undergo internalization in cycling, proteins that target intracellular localization and enhance intracellular half life, and the like.

Liposomes are commercially available from Life Technologies, Inc., for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications.

When administered, the α-synuclein or tau expression-modulating compounds (also referred to herein as therapeutic compounds and/or pharmaceutical compounds) of the present invention are administered in pharmaceutically acceptable preparations. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts. Preferred components of the composition are described above in conjunction with the description of the pharmacological agents and/or compositions of the invention.

A pharmacological agent or composition may be combined, if desired, with a pharmaceutically acceptable carrier. The term “pharmaceutically acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pharmacological agents of the invention, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride; chlorobutanol; parabens and thimerosal.

The therapeutics of the invention can be administered by any conventional route including injection or by gradual infusion over time. Various modes of administration will be known to one of ordinary skill in the art which effectively deliver the pharmacological agents of the invention to a desired tissue, cell, or bodily fluid. The administration methods include: topical, intravenous, oral, inhalation, intracavity, intrathecal, intrasynovial, buccal, intraperitoneal, sublingual, intranasal, transdermal, intravitreal, subcutaneous, intramuscular and intradermal administration. The invention is not limited by the particular modes of administration disclosed herein. Standard references in the art (e.g., Remington: The Science and Practice of Pharmacy, 19th edition, 1995) provide modes of administration and formulations for delivery of various pharmaceutical preparations and formulations in pharmaceutical carriers. Other protocols which are useful for the administration of pharmacological agents of the invention will be known to one of ordinary skill in the art, in which the dose amount, schedule of administration, sites of administration, mode of administration (e.g., intra-organ) and the like vary from those presented herein.

The therapeutic compositions may conveniently be presented in unit dosage form and may be prepared by any of the methods well known in the art of pharmacy. All methods include the step of bringing the compounds into association with a carrier that constitutes one or more accessory ingredients. In general, the compositions are prepared by uniformly and intimately bringing the therapeutic agent into association with a liquid carrier, a finely divided solid carrier, or both, and then, if necessary, shaping the product.

Compositions suitable for parenteral administration conveniently comprise a sterile aqueous preparation of the therapeutic agent, which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or diglycerides. In addition, fatty acids such as oleic acid find use in the preparation of injectables. Carrier formulations suitable for oral, subcutaneous, intravenous, intramuscular, etc. can be found in Remington's Pharmaceutical Sciences, Mack Publishing Company, Easton, Pa.

Compositions suitable for oral administration may be presented as discrete units such as capsules, cachets, tablets, or lozenges, each containing a predetermined amount of the therapeutic agent. Other compositions include suspensions in aqueous liquors or non-aqueous liquids such as a syrup, an elixir, or an emulsion.

The invention provides a composition of the above-described agents for use as a medicament, methods for preparing the medicament and methods for the sustained release of the medicament in vivo. Delivery systems can include time-release, delayed release or sustained release delivery systems. Such systems can avoid repeated administrations of the therapeutic agent of the invention, increasing convenience to the subject and the physician. Many types of release delivery systems are available and known to those of ordinary skill in the art. They include polymer-based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

In one particular embodiment, the preferred vehicle is a biocompatible microparticle or implant that is suitable for implantation into the mammalian recipient. Exemplary bioerodible implants that are useful in accordance with this method are described in PCT International application no. PCT/US95/03307 (Publication No. WO 95/24929, entitled “Polymeric Gene Delivery System”). PCT/US95/03307 describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the compound(s) of the invention is encapsulated or dispersed within the biocompatible, preferably biodegradable polymeric matrix disclosed in PCT/US95/03307. The polymeric matrix may be in the form of a microparticle such as a microsphere (wherein the compound is dispersed throughout a solid polymeric matrix) or a microcapsule (wherein the compound is stored in the core of a polymeric shell). Other forms of the polymeric matrix for containing the compounds of the invention include films, coatings, gels, implants, and stents. The size and composition of the polymeric matrix device is selected to result in favorable release kinetics in the tissue into which the matrix device is implanted. The size of the polymeric matrix device further is selected according to the method of delivery that is to be used. The polymeric matrix composition can be selected to have both favorable degradation rates and also to be formed of a material that is bioadhesive, to further increase the effectiveness of transfer when the device is administered to a vascular surface. The matrix composition also can be selected not to degrade, but rather, to release by diffusion over an extended period of time.

Both non-biodegradable and biodegradable polymeric matrices can be used to deliver agents of the invention of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the agents of the invention are delivered using the bioerodible implant by way of diffusion, or more preferably, by degradation of the polymeric matrix. Exemplary synthetic polymers that can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993:26:581-587, the teachings of which are incorporated herein by reference, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Use of a long-term sustained release implant may be particularly suitable for treatment of established neurological disorder conditions as well as subjects at risk of developing a neurological disorder. “Long-term” release, as used herein, means that the implant is constructed and arranged to deliver therapeutic levels of the active ingredient for at least 7 days, and preferably at least 30-60 days or more. The implant may be positioned at or near the site of the neurological damage or the area of the brain or nervous system affected by or involved in the neurological disorder. Long-term sustained release implants are well known to those of ordinary skill in the art and include some of the release systems described above.

Some embodiments of the invention include methods for treating a subject to reduce the risk of a disorder associated with abnormal levels of α-synuclein or tau expression. The methods involve selecting and administering to a subject who is known to have, is suspected of having, or is at risk of having an abnormal level of α-synuclein or tau expression, an α-synuclein or tau expression-modulating compound for treating the disorder. Preferably, the α-synuclein or tau expression-modulating compound is a compound for decreasing α-synuclein or tau expression and is administered in an amount effective to decrease α-synuclein or tau expression and therefore reduce production of α-synuclein or tau polypeptide.

Another aspect of the invention involves reducing the risk of a disorder associated with abnormal levels of α-synuclein or tau expression, by the use of treatments and/or medications to modulate levels of α-synuclein or tau expression thereby reducing, for example, the subject's risk of an α-synuclein- or tau-associated disorder.

In a subject determined to have an α-synuclein- or tau-associated disorder, an effective amount of an α-synuclein or tau expression-modulating compound is that amount effective to decrease levels of α-synuclein or tau expression in a subject and therefore reduce α-synuclein or tau production in the subject. For example, in the case of Alzheimer's disease an effective amount may be an amount that inhibits (reduces) the levels of α-synuclein or tau production.

A response to a prophylatic and/or treatment method of the invention can, for example, also be measured by determining the physiological effects of the treatment or medication, such as the decrease or lack of disease symptoms following administration of the treatment or pharmacological agent. Other assays will be known to one of ordinary skill in the art and can be employed for measuring the level of the response. For example, the behavioral and neurological diagnostic methods that are used to ascertain the likelihood that a subject has Alzheimer's disease, and to determine the putative stage of the disease can be used to ascertain the level of response to a prophylactic and/or treatment method of the invention. The amount of a treatment may be varied for example by increasing or decreasing the amount of a therapeutic composition, by changing the therapeutic composition administered, by changing the route of administration, by changing the dosage timing and so to on. The effective amount will vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner.

The factors involved in determining an effective amount are well known to those of ordinary skill in the art and can be addressed with no more than routine experimentation. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

The therapeutically effective amount of a pharmacological agent of the invention is that amount effective to modulate α-synuclein or tau expression and reduce, prevent, or eliminate an α-synuclein- or tau-associated disorder. Additional tests useful for monitoring the onset, progression, and/or remission, of an α-synuclein- or tau-associated disorder such as those described above herein, are well known to those of ordinary skill in the art. As would be understood by one of ordinary skill, for some disorders (e.g. Alzheimer's disease, Parkinson's disease) an effective amount would be the amount of a pharmacological agent identified using an assay of the invention that decreases the levels of α-synuclein or tau expression to a level and/or activity that diminishes the disorder, as determined by the aforementioned tests.

In the case of treating a particular disease or condition the desired response is inhibiting the progression of the disease or condition. This may involve only slowing the progression of the disease temporarily, although more preferably, it involves halting the progression of the disease permanently. This can be monitored by routine diagnostic methods known to one of ordinary skill in the art for any particular disease. The desired response to treatment of the disease or condition also can be delaying the onset or even preventing the onset of the disease or condition.

The pharmaceutical compositions used in the foregoing methods preferably are sterile and contain an effective amount of a pharmacological agent for producing the desired response in a unit of weight or volume suitable for administration to a patient.

The doses of pharmacological agents administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent of the invention may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days.

Administration of pharmacological agents of the invention to mammals other than humans, e.g. for testing purposes or veterinary therapeutic purposes, is carried out under substantially the same conditions as described above. It will be understood by one of ordinary skill in the art that this invention is applicable to both human and animal diseases including α-synuclein- or tau-associated disorders of the invention. Thus, this invention is intended for use in husbandry and veterinary medicine as well as in human therapeutics.

The invention will be more fully understood by reference to the following examples. These examples, however, are merely intended to illustrate the embodiments of the invention and are not to be construed to limit the scope of the invention.

### EXAMPLES

### Example 1

### Introduction

We have examined the function of α-synuclein to develop therapeutic strategies to address the pathology of Parkinson's disease. Our results indicate that α-synuclein has a role in iron homeostasis. We have determined that there are putative iron-responsive element (IRE) sequences having RNA secondary structure in a 52 base (SEQ ID NO:1) 5′ untranslated region (5′UTR) of α-synuclein mRNA. By predicting this RNA secondary structure we can screen for RNA targeted drugs that limit the translation of α-synuclein (ASN) driven from the 52 base α-synuclein 5′ untranslated region as a therapeutic strategy for Parkinson's disease.

### Methods

Quantitative Real-Time PCR (qRTPCR)

Quantitative real-time PCR (qRTPCR) was used to measure steady-state mRNA levels for α-synuclein mRNA relative to transferrin receptor (TfR) mRNA levels in response to iron. Neuroblastoma (SY5Y) cells were treated with iron or desferrioxamine (DFO) for 6 hours or 16 hours and control cells were left untreated (U) for the same indicated times (i.e., 6 hours untreated=U6, and 16 hours untreated=U16). TfR mRNA was the experimental positive control mRNA.

The qRTPCR was conducted as follows. Total RNA was isolated from iron and desferrioxamine treated neuroblastoma cell line, SH-SY5Y according to manufacturers specification (RNAeasy Mini Kit, Qiagen, Valencia, Calif.). The optional on-column DNase digestion with the RNase-Free DNase set (Qiagen) was incorporated into all RNA isolations.

3 μg total RNA was primed with oligo-dT and the Superscript II enzyme according to manufacturer's instructions (Invitrogen, Carlsbad, Calif.) to synthesize 100 μg cDNA. For qRTPCR, primer specificity was initially verified by searching for homology to genomic databases using NCBI BLAST. After completion of qRTPCR, the appropriately sized amplicon were confirmed by agarose gel electrophoresis. All reactions were performed in duplicate in a 50 μl volume consisting of 3 μl cDNA, 15 μmol each of the forward and reverse primer of β-Actin and 45 μmol each of the forward and reverse primer of all other primer pairs, and the fluorescent SYBR Green I dye. Fluorescence was measured at the end of each annealing cycle using the ABI Prism 7000 (Applied Biosystems, Foster City, Calif.). Average threshold cycle (Ct) values were measured in the exponential phase of the PCR reaction for each duplicate sample. Average Ct values were normalized against that β-Actin amplified in parallel from the same sample. Relative expression of the treated samples was determined by first normalizing against that of the untreated samples (ΔC_{T}^{sample}−ΔC_{T}^{normal}=ΔΔC_{T}) and then calculating the fold change (fold change=2^{-ΔΔCt}).

Primers used for quantitative real-time PCR were as follows:

β-Actin
[forward, 5′-CACCTTCTACAATGAGCTGCG-3′ (SEQ ID NO:
9); reverse,5′-GCACAGCCTGGATAGCAACG-3′ (SEQ ID
NO: 10)],
Transferrin Receptor
[forward 5′-AGGAGCCAGGAGAGGACTTCC-3′ (SEQ ID NO:
11); reverse, 5′-TCTCCGACAACTTTCTCTTCAGG-3′ (SEQ
ID NO: 12)],
ASN
[forward, 5′-TGACGGGTGTGACAGCAGTAG-3′ (SEQ ID NO:
13); reverse, 5′-AGCCAGTGGCTGCTGCA-3′ (SEQ ID NO:
14)].

The details of the annealing temperature are as follows. For real-time PCR calculation of iron/desferrioxamine-induced levels of ASN mRNA relative to TfR mRNA to (positive control for DFO/ferric ammonium citrate (FAC)-induced iron growth conditions), cDNA was synthesized with the Omniscript RT Kit (Qiagen) using 2 μg total RNA in a 20 μl reaction volume. For real-time PCR, the cDNA was amplified using an ABI PRISM 7000 Sequence Detection System (PE Applied Biosystems, Foster City, Calif.). The dsDNA-specific dye SYBR Green I incorporated into the PCR reaction buffer QuantiTech™ SYBR PCR (Qiagen) to allow for quantitative detection of the PCR product in a 25-μl reaction volume. The temperature profile of the reaction was 95° C. for 10 min, 40 cycles of denaturation at 95° C. for 15 sec, annealing at 60° C. for 30 sec, and extension at 72° C. for 30 sec. An internal housekeeping gene control, β-actin, was used to normalize differences in RNA isolation, RNA degradation, and the efficiencies of the RT. The size of the PCR product was first verified on a 1.5% agarose gel, followed by melting curve analysis.

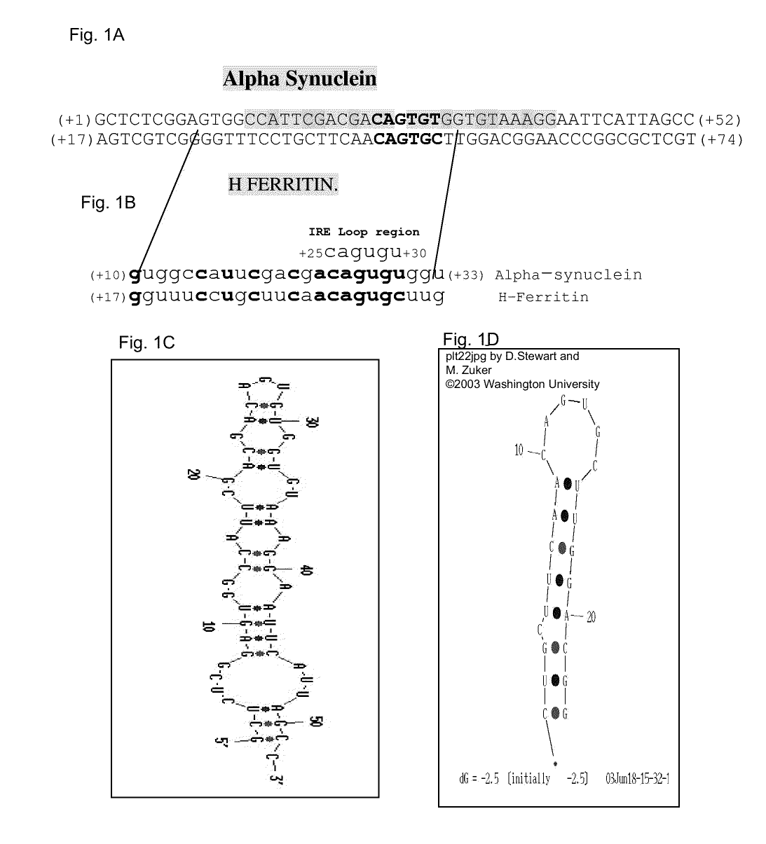

We generated a model of the folding of the α-synuclein mRNA 5′ untranslated region using the fold program mfold at the website of Dr. Michael Zuker and his colleagues at The Bioinformatics Center at Rensselaer and Wadsworth (www.bioinfo.rpi.edu/˜zukerm/). The predicted model is illustrated in FIG. 1. Interestingly the apex of the predicted stemloop was found to align with the loop region of the canonical iron responsive element (IRE) in ferritin and transferrin receptor (TfR) mRNAs. The 52 base α-synuclein 5′ untranslated region (SEQ ID NO:1) was computer predicted to form a unique RNA stemloop wherein the RNA sequences in the loop region sequence encode a CAGUGU motif that was aligned with the 5′CAGUGC3′ loop at the apex of the loop of the H-Ferritin mRNA IRE stemloop. The canonical IRE in transferrin receptor and ferritin mRNAs has been well characterized to encode the loop region sequence as 5′CAGUGN3′ that is found in several IREs encoded by the transcripts of genes involved in iron metabolism.

Using the public RNA fold program we computer-scanned the 52 base 5′ untranslated region (5′UTR) of α-synuclein mRNA to determine for the presence of putative iron-responsive Elements (IRE). (Zuker, M. 2003 Nucleic Acids Res 31, 3406-3415). We discovered the presence of a sequence element in the predicted loop region at the apex of the secondary structure folded from α-synuclein 5′UTR sequences (+25−30) which was identical to the loop region of the 5′UTR IRE stemloop in H-ferritin mRNA (+43−48). FIG. 1 shows the presence of this canonical 5′CAGUGN3′ in both the ferritin and α-synuclein mRNA 5′ untranslated regions.

We determined that the RNA structure formed from the α-synuclein 5′UTR was a strong candidate sequence element to harbor a translational enhancer activity that controls α-synuclein mRNA translational efficiency and the steady-state levels of intracellular α-synuclein levels in the cells, regulated at both the baseline level and in response to iron. We made α-synuclein-specific constructs and tested whether the α-synuclein 5′ untranslated region harbored an iron-responsive element as was previously found to be present in the Alzheimer's APP transcript (Rogers J. T., et al., 2002 J Biol Chem 277:45518-45528, U.S. Pat. Nos. 6,310,197 and 6,849,405, each of which is incorporated herein by reference). The single RNA stemloop formed from α-synuclein 5′UTR sequences is shown in FIG. 1.

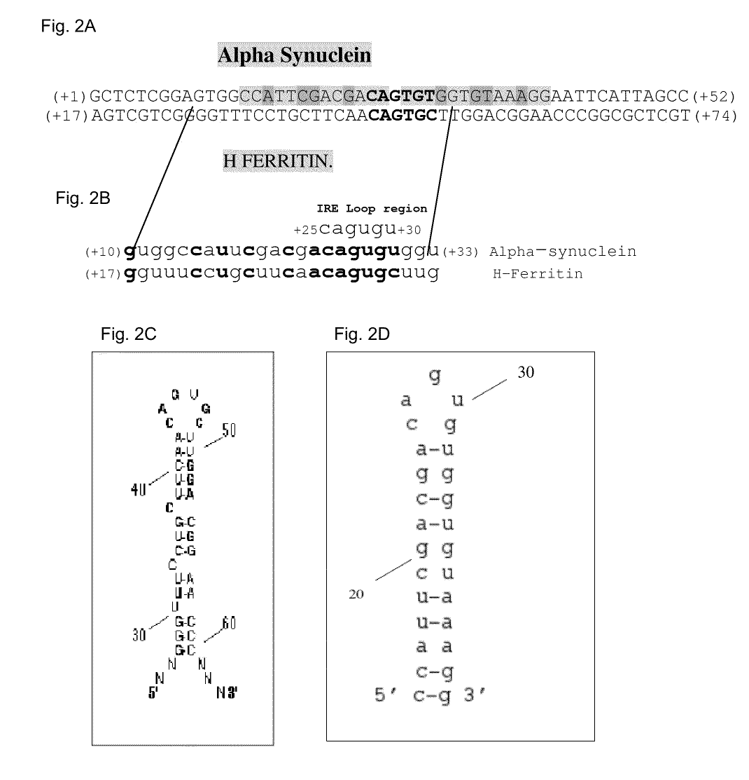

We generated an alternative α-synuclein 5′ UTR specific stemloop by manually pairing bases that best fit a stem structure around the reference point CAGUGC (from the ferritin mRNA illustrated in FIG. 2C) in the α-synuclein mRNA 5′ untranslated region as is illustrated in FIG. 2D.

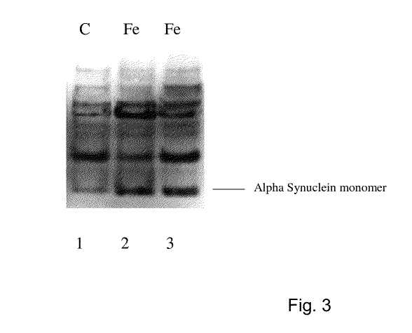

We determined that an active IRE was present in the α-synuclein 5′UTR and we predicted that α-synuclein protein might exhibit the same pattern of iron responsive expression as occurs for the ferritin subunits. We found that treatment of SY5Y cells with iron (FAC) resulted in an increase in α-synuclein protein expression. FIG. 3 shows results with untreated SY5Y cells in lane 1, FAC-treated for 6 hr (lane 2), and FAC-treated for 3 days in lane 3. The FAC treatment was at a concentration of 100 μM FAC. Additionally, we found that short-term (6h) and long-term (3d) treatment of SY5Y cells with desferrioxamine caused a dramatic reduction in the intracellular levels of α-synuclein protein.

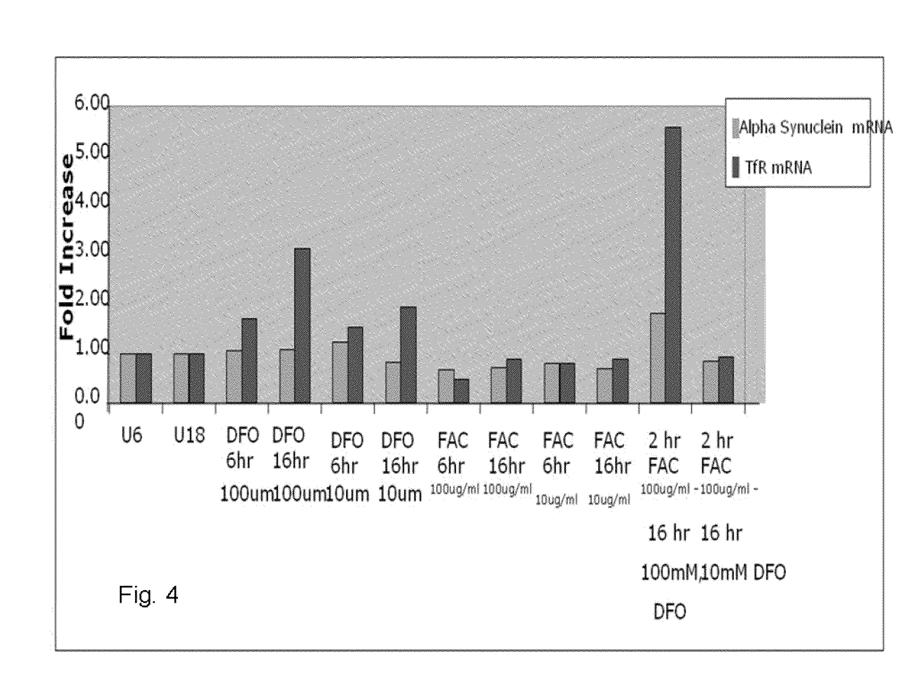

In addition to identifying that desferrioxamine modulated the levels of α-synuclein protein we employed real-time PCR (RT-PCR) analysis on RNA purified from similarly treated SY5Y neuroblastoma cells to measure α-synuclein mRNA levels (FIG. 4). FIG. 4 illustrates qRT-PCR for ASN mRNA levels in response to changed iron levels in SY5Y cells relative to TfR mRNA. The TfR-mRNA levels were two-fold decreased by ferric ammonium citrate (FAC) treatment for 6 hours whereas alpha synuclein mRNA did not change as significantly although it was reduced. The figure shows that DFO increased TfR mRNA levels but left alpha synuclein expression unaltered. When considered in the light of the Western (protein levels) in FIG. 3, our data suggested translational control of alpha synuclein by acute iron administration to neuroblastoma cells. We found that α-synuclein mRNA levels were unchanged under conditions of intracellular iron chelation with desferrioxamine (DFO). We also found that TfR mRNA steady-state levels were 3-fold up-regulated by desferrioxamine (DFO) treatment (SY5Y cells at 100 μM). The finding that TfR mRNA steady-state levels were upregulated by intracellular iron chelation confirmed the use of TfR mRNA levels as an positive control index for intracellular iron chelation (Klausner, R. D., et al., 1993 Cell 72:19-28).

FIG. 4 also indicates the results of real-time PCR analysis demonstrating that iron chelation with DFO increased transferrin receptor (TfR) mRNA levels. We determined that iron influx decreased TfR mRNA levels relative to beta actin, which was unresponsive to changed metal treatment of neuroblastoma cells. Thus, the fold changes in TfR and alpha synuclein transcripts were relative to beta actin mRNA levels. Iron influx was examined with the addition of FAC and iron chelation was examined with the addition of DFO, and neither changed the steady-state levels of α-synuclein transcript. These results are illustrated in FIG. 4, which shows the results of quantitative real-time PCR and indicates that α-synuclein mRNA levels were not modulated by iron chelation with DFO or iron influx with FAC.

Transferrin receptor mRNA (TfR mRNA) was the experimental positive control mRNA, and we determined that TfR mRNA was up-regulated by 3× fold in response to DFO and down-regulated 2× fold by FAC. Neuroblastoma (SY5Y) cells were treated with each (or both) agents (at several concentrations) for 6 hours or 16 hours and control cells were left untreated (U) for the same indicated times (i.e., 6 hours untreated=U6, and 16 hours untreated=U16).

### Discussion

The results indicated that: (i) Steady-state levels of α-synuclein protein were reduced in SY5Y neuroblastoma cells during conditions of intracellular iron chelation with 100 μM DFO (16 h), (ii) at the same time α-synuclein mRNA levels were unchanged in response to DFO treatment [TfR mRNA levels were changed as a positive control to verify that we effectively chelated iron from SY5Y cells from our experimental conditions (Klausner, R. D., et al., 1993 Cell 72:19-28)], (iii) this pattern of regulation was consistent with our discovery of a putative iron responsive element in the 5′UTR of α-synuclein mRNA.

In neuroblastoma cells we found that α-synuclein protein expression was reduced under conditions of intracellular iron chelation (DFO treatment). This inhibition of α-synuclein by desferrioxamine treatment of SY5Y cells appeared to be caused by a selective block of α-synuclein mRNA translation. This finding supported the presence of an iron-responsive element in the α-synuclein 5′UTR as we predicted.

### Use of an α-Synuclein 5′ Untranslated Region:

The 52 base 5′UTR of α-synuclein (SEQ ID NO:1) folds into a distinct RNA secondary structure (FIG. 1). We previously screened the Alzheimer's APP 5′UTR as a drug target for AD therapeutics (Payton, S., et al., 2003 J Mol Neurosci. 20(3):267-75 and U.S. Pat. Nos. 6,310,197 and 6,849,405, each of which is incorporated herein by reference). We have performed further transfection-based assays to employ the α-synuclein 5′UTR as a valid drug target for the development of therapeutic agents for the treatment of Parkinson's disease (PD). The α-synuclein 5′UTR presents a drug target for PD therapeutics. An effective drug screen to the α-synuclein 5′UTR (luciferase assay) was used to identify novel chelators and antioxidant drugs that address other underlying features of PD (i.e., anti oxidants that limit α-synuclein expression but also protect neurons from oxidative stress in their own capacity derived from inhibiting neurotoxic iron catalyzed by Fenton-based chemical reactions).

Our findings suggested the presence of an active iron-responsive element in the α-synuclein 5′UTR that would account for the observed iron dependent translational control of α-synuclein (FIGS. 3 and 4). Like ferritin, α-synuclein expression appears to be controlled at the translational level by an active 5′ untranslated region iron-responsive element.

### Example 2

Tau mRNA Regulation by Iron-responsive Elements, a new RNA directed target for AD-based Therapeutics to prevent Neurofibrillary Tangle formation.

### BACKGROUND

Tau is a microtubule-associated protein that is intimately associated with the neurofibrillary tangles of AD (Kosik, K. S., et al., 2002 J. Mol. Neurosci, 19, 261-6). Missplicing of a 4 repeat stretch by alternative Tau mRNA splicing is associated with frontal temporal dementia (Kosik, K. S., et al., 2002 J. Mol Neurosci, 19, 261-6). Altered expression and pathology of Tau is also intimately linked in Supranuclear Atrophy palsy (Kosik, K. S., et al., 2002 J. Mol Neurosci, 19, 261-6) and the iron chelator ferralex prevented Tau tubule formation (Shin et al., 2002 Brain Res. 961:139-46). We have identified three Iron-regulatory Elements (IREs) in the Tau mRNA 3′UTR and two counterparts in the coding region of tau (Bioinformatic search of the human Tau genes (NCBI)) and have used these tau 3′UTR sequences as a valid drug target for developing treatments for AD.

Recent evidence has shown that induction of heat shock protein (HSP) limited Tau protein levels and therapeutically reduced formation of neurofibrillary tangles (Wallace et al., 1993 Brain Res Mol Brain Res 1993 19(1-2):140-8; Shimura, H., et al., 2004 J Biol Chem. 279(6):4869-76; Dou, F., et al., 2003 Proc Natl Acad Sci USA 100(2):721-6). We investigated methods of screening drugs directed to the 3′UTR of Tau mRNA for identifying small molecules that destabilize the Tau transcript sufficient to limit Tau production and therefore provide less template for Abeta (Aβ) induced formation of neurofibrillary tangles. Tau-deficient mice are viable (Dawson, H. N., et al., 2001 J Cell Sci. 114:1179-87), and the lack of long-term toxic or deleterious consequences that result from reduced levels of tau supports the tau production-limiting strategies we have developed. Thus, a drug that depletes neurons of tau would make the neurons less likely to develop neurofibrillary tangles and validates the methods we have developed for using tau 3′UTR sequences as a valid drug target for AD.

### Methods

Quantitative real-time PCR (QRTPCR) was performed on RNA extracted from iron/zinc and desferrioxamine treated mRNA. Methods for QRTPCR were as described for priming an α-synuclein amplicon (see Example 1) except that oligonucleotides were (NM016835Rev) 5′TCGACTGGACTCTGTTCTTGA3′ (SEQ ID NO:15) and (NM 016835For) 5′TGGCCAGGTGGAAGTAAAATC3′ (SEQ ID NO:16).

Quantitative real-time PCR data was performed and demonstrated that that Tau mRNA levels are up-regulated by iron chelation with desferrioxamine (DFO) and down regulated after iron influx with ferric ammonium citrate (FAC). Neuroblastoma cells (SY5Y) were treated with each agent for 6 hours or 16 hours and control cells were left untreated (U) for the same indicated times (i.e., 6 hours untreated=U6, and 16 hours untreated=U16). Transferrin receptor mRNA (TfR mRNA) was the experimental positive control mRNA which was up-regulated by 4.5× fold in response to DFO and 4-fold reduced by FAC.

### Results

Using the publicly available RNA fold program of Dr. Michael Zuker and colleagues at www.bioinfo.rpi.edu/applications/mfold/, we computer scanned the 3′ untranslated region (3′UTR) of Tau mRNA to determine for the presence of putative Iron-responsive Elements (IREs) (Zuker, M. 2003 Nucleic Acids Res 31, 3406-3415). We discovered the presence of a sequence element in the predicted loop region at the apex of three stemloops folded from tau mRNA 3′UTR sequences which was identical to the CAGUGN iron-responsive element (IRE) motif in ferritin and transferrin receptor mRNAs.

The 3′UTR IREs control the RNA stability of the transferring receptor (TfR mRNA) in response to cellular iron levels (Leedman, P., et al., 1996 J Biol Chem 271:12017-12023). We determined that the RNA structure formed from the Tau 3′UTR is a strong candidate sequence element to harbor a RNA stability activity that controls Tau mRNA steady-state levels in the neuronal cells, regulated at both the baseline level and in response to iron.

We employed real-time PCR (RT-PCR) to demonstrate that iron chelation with desferrioxamine modulated the levels of Tau mRNA. RNA purified from SY5Y neuroblastoma cells was employed to measure Tau mRNA levels. Tau mRNA levels were dramatically changed under conditions of intracellular iron chelation with desferrioxamine (DFO) at the same time that TfR mRNA steady state levels were 3-fold up-regulated by DFO treatment (SY5Y cells at 100 μM DFO). TfR mRNA steady state levels are regulated by intracellular iron chelation confirming the use of TfR mRNA levels to serve as an positive control index for intracellular iron chelation (Klausner, R. D., et al., 1993 Cell 72:19-28).

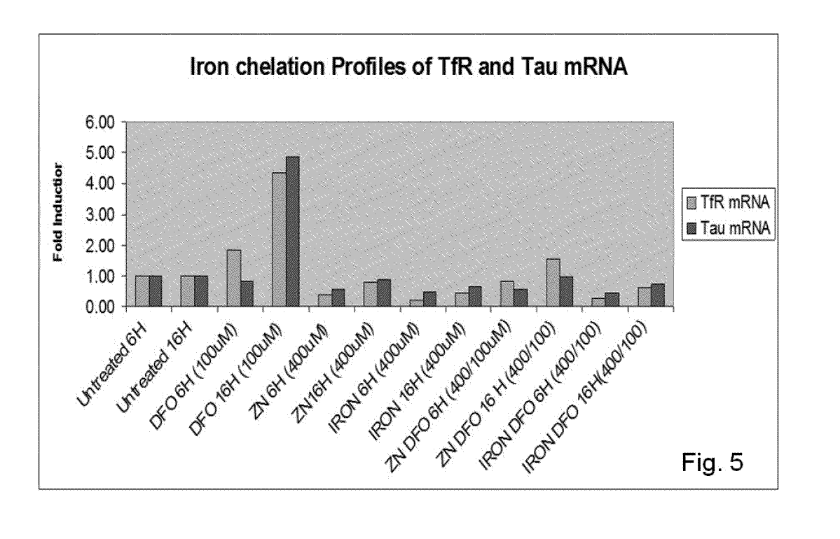

FIG. 5 shows quantitative real-time PCR data showing that Tau mRNA levels are up-regulated by iron chelation with desferioxamine (DFO) and down regulated after iron influx with ferric ammonium citrate (FAC). Transferrin receptor mRNA (TfR mRNA) was the experimental positive control mRNA which was up-regulated by 4.5× fold in response to DFO and 4-fold reduced by FAC.

The results shown in FIG. 5 demonstrate that there was 4.5-fold increase in induction for TfR mRNA, 5-fold increase in induction for Tau mRNA in response to 16 hours treatment with DFO. The fold changes in tau transcripts were relative to beta actin mRNA levels. Inverse regulation of tau mRNA was observed in the presence of iron (a 0.66 fold reduction of for Tau mRNA at 400 micromolar iron (16 h), at the same time there was a 0.45 fold reduction for TfR mRNA (FIG. 5). FIG. 5 shows results of qRT-PCR for tau mRNA levels in response to changed iron levels in SY5Y cells relative to TfR mRNA. Zinc was used as a control for iron specificity. The results indicated that TfR mRNA was more responsive to iron than zinc but that TfR mRNA was surprisingly responsive to zinc. It was also determined that the pattern of tau expression by iron/zinc changes reflected TfR mRNA changes. This was consistent with the proposal that IRE-like RNA stemloops in the tau mRNA 3′UTR are post-transcriptional control points that determine tau protein levels by iron (Like TfR), and that these RNA structure can be used as drug targets to identify small molecules that can be used to limit tau production.

### Discussion

Because the general pattern of IREs in the TfR and Tau mRNAs demonstrates 3′UTR IREs (5 for TfR mRNA and 3 IREs for Tau mRNA) in each case, we have examined whether Tau mRNA levels are regulated by iron at the level of message stability as is the case for TfR mRNA (Klausner, R. D., et al., 1993 Cell 72:19-28). We have investigated use of a drug-targeting program to suppress Tau mRNA 3′UTR and decrease tau message stability as a method of identifying compounds that are therapeutic for AD. We have developed methods of targeting the Tau mRNA 3′UTR that provide a valid strategy—a strategy that is also supported by the finding that the loss of Tau function in Tau knock-out mice is not lethal as the function appears to be replaced by other microtubule associated proteins.

### Example 3

Immunoprecipitation RT-PCR (IP RTPCR) experiments were performed using SH-SY5Y neuroblastoma cell lysates to determine specific binding to Iron—Regulatory Protein-1 (IRP-1). α synuclein (ASN) is a 15 kDa protein (monomer) that is ubiquitously expressed in all tissues. However aggregates of ASN cause a profound neurotoxicity in the neurons of the substantia nigra of Parkinson's Disease patients (Sulzer, D. 2001 Nat. Med. 7:1280-1282). There are recessive mutations in the a synuclein protein that cause early onset genetic forms of Parkinson's disease (Papadimitriou, A., et al., 1999 Neurology 52:651-654). Knowledge as to the function of α-synuclein will help in the development of therapeutic strategies to address the pathology of Parkinson's disease. α-synuclein has been found to be up-regulated during MPP+-induced apoptosis in neuroblastoma cells, and transferrin receptor, iron and hydrogen peroxide were intermediates (Kalivendi, S. V., et al. (2004). J Biol Chem 279, 15240-15247). We have investigated the role of α-synuclein in iron homeostasis. Iron homeostasis has been proposed to be a causative agent propelling neuronal death during the course of PD (Faucheux, B. A., and Hirsch, E. C. (1998). Ann Biol Clin (Paris) 56 Spec No, 23-30).

We have identified the presence of Iron responsive Element (IRE) sequences in the 52 base 5′ untranslated region (5′UTR) of α-synuclein mRNA. By predicting this RNA secondary structure have developed assays to screen for RNA targeted drugs that limit the translation of ASN driven from the 52 base ASN 5′ untranslated region as a therapeutic strategy for Parkinson's disease. The identification of the role of IRE allowed us to use an approach that is similar to the strategy we used to screen for anti-amyloid Alzheimer's disease agents (Rogers J T, et al., (2002) J Mol Neurosci 19, 77-82; Payton, S., et al., 2003 J Mol Neurosci. 20(3):267-75).

### Methods

Immunoprecipitation RT-PCR (IP RTPCR) was performed using standard methods and the results demonstrated that α-synuclein mRNA was immunopreciptated from SH-SY5Y neuroblastoma cell lysates specifically bound to Iron—Regulatory Protein-1 (IRP-1). Neuroblastoma cells were treated with (i) DFO (50 mM), (ii) feTf (10 μM) or remained untreated. Cells were lysed in RIPA buffer, and cytosolic extracts were immunoprecipitated with antibody to IRP-1 and IRP-2. After washing with Fe-beads the bound mRNA or superatant RNA was reverse transcribed and the mRNAs attached to the IRP-1/IRP_{—}2 were identified by immunoprecipitation of RNA that selectively interacted with IRP-1/IRP-2 (method of Thomson, A. M., et al., Biol. Chem. 2005 Aug. 26; 280(34):30032-45. Epub 2005 Jun. 20.

### Results

We investigated the RNA structure formed from the α-synuclein 5′UTR as a candidate sequence element to harbor a translational enhancer activity that controls ASN mRNA translational efficiency and the steady-state levels of intracellular α-synuclein levels in the cells, regulated at both the baseline level and in response iron. ASN-specific constructs were made to test whether the ASN 5′ untranslated region harbors an Iron-responsive Element. The single RNA stemloop formed from ASN 5′UTR sequences is shown in FIG. 1 (Zuker, M. (2003) Nucleic Acids Res 31, 3406-3415).

Assuming the presence of an active IRE in the ASN 5′UTR we predicted that α-synuclein might exhibit the same pattern of iron responsive expression as occurs for the ferritin subunits. Indeed we have found that short-term (6 h) and long-term (3d) treatment of SY5Y cells with desferrioxamine caused a dramatic reduction in the intracellular levels of α-synuclein protein.

At the same time that iron was observed to modulate the levels of α-synuclein protein we employed real-time PCR (RT-PCR) analysis on RNA purified from iron and desferrioxamine (iron chelator) treated SY5Y neuroblastoma cells to measure α-synuclein mRNA levels (FIG. 4). α-synuclein mRNA levels were unchanged under conditions of intracellular iron chelation with desferrioxamine at the same time that TfR mRNA steady state levels were 3-fold up-regulated by DFO treatment (SY5Y cells at 100 μM). TfR mRNA steady state levels are regulated by intracellular iron chelation confirming the use of TfR mRNA levels to serve as an positive control index for intracellular iron chelation (Klausner, R. D., et al., (1993) Cell 72, 19-28).

The regulation of α-synuclein resembled the translational control of ferritin mRNA in that altered iron levels leave L- and H-subunit transcript levels unchanged but iron induced levels of protein. Ferritin mRNA is known to be regulated by the translational repression of L- and H-mRNAs when Iron-regulatory Proteins (IRP-1/IRP-2) bound to 5′ untranslated region Iron responsive Element (IRE) stem-loops (Thomson, A. M., et al., (2005) J Biol Chem 280, 30032-30045). Here iron influx released IRP-1/IRP-2 binding and the ferritin subunits (19,000 dalton, 21,000 dalton) were translated at an increased rate.

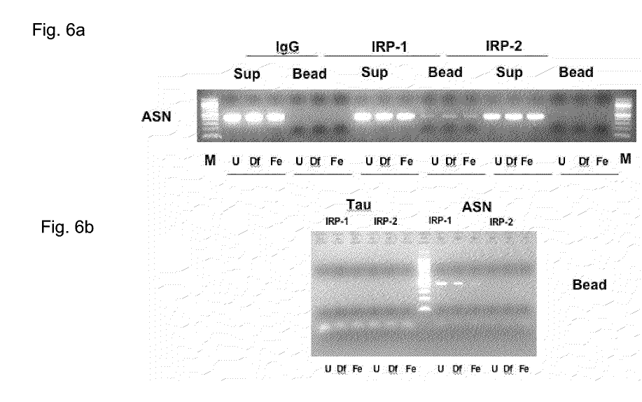

We tested whether α-synuclein mRNA interacted with IRP-1 or IRP-2 by use of an RNA co-immunoprecipitation technique shown in FIG. 6. In this IPRT-PCR experiment α-synuclein mRNA was selectively co-immunoprecipitated with IRP-1 from neuroblastoma lysates (the presence of ASN mRNA was detected by PCR of cDNA reverse transcribed from mRNA immunopreciptated with IRP-1 on washed beads according to standard methods (Thomson, A. M., et al., (2005) J Biol Chem 280, 30032-30045). Ferritin mRNA was selectively immunopreciptated with both IRP-1 and IRP-2, whereas α-synuclein mRNA was selectively bound to IRP-1, not IRP-2. This technique was semi qualitative PCR, though the amount of α-synuclein mRNA attached IRP-1 was iron dependent in the experiment shown in FIG. 6B. To test the presence of genuine IRE in the α-synuclein 5′ untranslated region FIG. 6 provides data that ASN mRNA interacts with Iron-regulatory Protein-1 (IRP-1) by use of an IRP-1/IRP-2 IPRTPCR experiment. Our data confirmed that 5′ untranslated region sequences in ASN mRNA indeed are iron responsive (FIG. 6). The interaction of IRP-1 with the ASN 5′UTR is also tested by RNA gel shift analysis and biotin probe pull-down experiments. The results (see FIG. 6B) showed that IRP-1 binding was reduced in iron-treated SH-SY5Y cells correlated with increased α-synuclein protein production during iron influx.

### Example 4

### pGL3 Construct Generation, Transient Transfection, FE+DFO Treatment and Luciferase and Green Fluorescent Protein (GFP) Assays

To determine whether α-synuclein 5′UTR sequences were iron responsive SH-SY5Y transfection experiments were performed using an ASN 5′UTR pGL3 construct compared to pGL3, and control vector in which the luciferase expression was translated in the absence of the α-synuclein 5′UTR.

### Methods

A 46 base sequence (SEQ ID NO:21) of the α-synuclein 5′ UTR was synthesized with a HindIII site at the 5′ end and a Nco I site at the 3′ end (Integrated DNA Technologies, Coralville, Iowa) and then ligated into a pGL-3 vector (Promega, Madison Wis.) immediately in front of the luciferase gene, under the control of a SV40 promotor. Wild-type pGL3 or pGL3 containing the α-synuclein 5′ UTR insert upstream of the luciferase promotor (α-synuclein 5′UTR-pGL3) were used. Expression of green fluorescent protein (GFP) within the pGL3 vector, controlled by a SV40 late poly(A) signal was used to control for transfection efficiency. 8×10⁵ neuroblastoma HYSY cells were seeded for 24 hours in 60-mm² dishes with 5 ml Dulbeccco's modified medium containing 10% fetal bovine serum. For transfection, cells were first rinsed 3× in Phosphate Buffered Saline (PBS) and then treated with 5 μg of purified DNA combined with PolyFect transfection reagent and growth medium, according to the manufacturer's specifications (Qiagen). After a 24 hours incubation at 37° and 5% CO₂ to maximize transfection efficiency, cells were washed in PBS 3×, trypsonized and plated to 95% confluence in 96-well plates. Cells were allowed to seed in the 96-well plates for 12 hours in Dulbecco's modified medium containing 10% fetal bovine serum and ampicillin, to select for cells containing the plasmid, which contained an ampicillin resistance gene. Treatment medium with various concentrations of FAC and DFO was prepared from serum-free Dulbenco's modified medium and stock solutions of 10 mg/ml FAC and 30 mM DFO. Treatment medium was prepared fresh the day of experiment and incubated for 1 hour at 37° prior to the experiment to maximize solubility. Media containing various concentrations of FAC or DFO were added to wells in a 96-well plate, with treatments performed in triplicate. After 48 hours of treatment with FAC or DFO, cell viability was confirmed by microscopic examination of each well. Cells were lysed with 1× Bright-glo luciferase assay buffer and the lysates combined with bright-glo luciferase assay substrate warmed to 37°, according to the specifications of the manufacturer (Promega, Madison, Wis.). All experiments were performed in triplicate. Automated luciferase assays were performed with a Wallac 1420 multi-label counter. GFP expression was quantified at 489/509 nm with the same Wallac 1420 counter to control for transfection efficiency. The α-synuclein 5′ UTR was found to confer Fe and DFO dependent translation of the luciferase reporter gene in a dose-dependent manner. With luciferase translation controlled by the α-synuclein 5′ UTR, treatment with 10 mg/ml FAC induced a 5-fold increase in luminescence, while to treatment with 100 mg/ml induced a 20 fold increase (p<0.001). Also with luciferase translation controlled by the α-synuclein 5′ UTR, 50 mM DFO decreased luminescence by 25% and 100 mM DFO decreased luminescence by 50% (p<0.001). FAC and DFO had no effect on luciferase activity with the wild-type pGL3 vector. We conclude that the α-synuclein mRNA contains a functional IRE in its 5′ UTR that confers translational regulation through an iron-dependent interaction with IRP1.

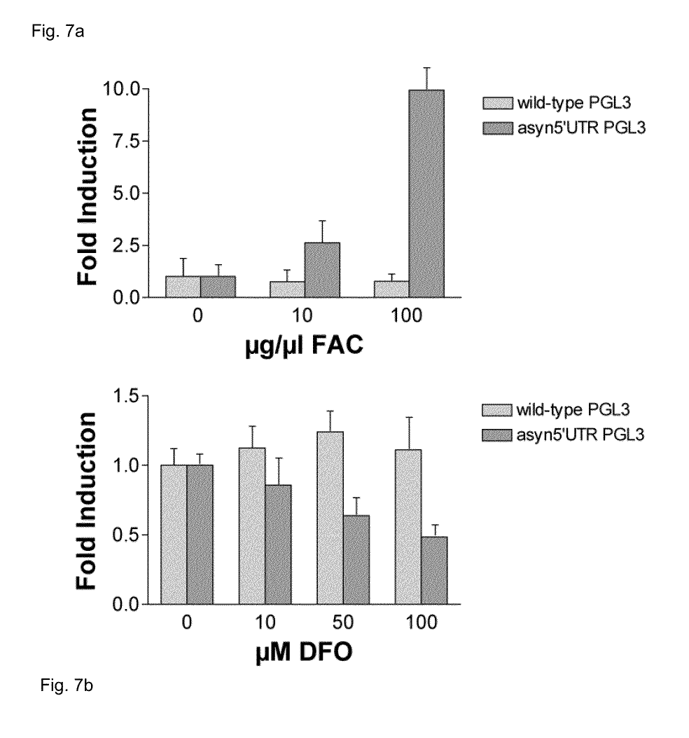

FIG. 7A shows the pGL3 vector in which the promotor for the luciferase reporter gene is SV40. The GFP is driven by a SV40 late poly(A) signal. FIG. 7B shows results indicating that the α-synuclein 5′ UTR conferred iron dependent activation and inhibition of a luciferase reporter gene. The α-synuclein 5′ UTR was found to confer Fe and DFO dependent translation of the luciferase reporter gene in a dose dependent manner. With luciferase translation controlled by the α-synuclein 5′ UTR, treatment with 10 mg/ml FAC induced a 5 fold increase in luminescence, while treatment with 100 mg/ml induced a 20 fold increase (p<0.001). Also with luciferase translation controlled by the α-synuclein 5′ UTR, 50 mM DFO decreased luminescence by 25% and 100 mM DFO decreased luminescence by 50% (p<0.001). FAC and DFO had no effect on luciferase activity with the wild-type pGL3 vector.

### Discussion

The overall conclusion from the experiments are:—(i) Steady-state levels of ASN were reduced in SY5Y neuroblastoma cells during conditions of intracellular iron chelation with 100 μM DFO (16 h), (ii) at the same time α-synuclein mRNA levels were unchanged in response to DFO treatment (TfR mRNA levels were changed as appositive control to verify that we effectively chelated iron from SY5y cells from our experimental conditions (Klausner, R. D., et al., (1993) Cell 72, 19-28). (iii) this pattern of regulation is consistent with our discovery of a putative iron responsive element in the 5′UTR of ASN mRNA.

In neuroblastoma cells we found that α-synuclein expression was activated under conditions of intracellular iron influx (and reduced when iron is chelated with desferrioxamine). The results suggested that the inhibition of SH-SY5Y ASN expression after desferrioxamine treatment may cause a selective block of ASN mRNA translation.

### Use of the α-Synuclein 5′ Untranslated Region:

We have generated stable neuroblastoma SH-SY5Y transfectants that express luciferase under the translational control of the 52-nucleotide α-synuclein mRNA 5′UTR and to green fluorescent protein (GFP) driven by a SV40 late poly(A) signal. The 52 base 5′UTR of α-synuclein folds into a distinct RNA secondary structure (FIG. 1). Now we have shown transfection-based assays that the ASN 5′UTR is selectively inhibited by the iron chelator desferrioxamine (FIG. 7). The results indicate that the ASN 5′UTR can be employed as a valid drug target for the development of therapeutic agents (other than desferrioxamine) for the treatment of Parkinson's disease.

We predicted that the α-synuclein 5′UTR presents a drug target for PD therapeutics. An effective drug screen to the ASN 5′UTR (luciferase assay) identifies novel chelators and antioxidant drugs that address other underlying features of PD (i.e., anti oxidants that limit ASN expression but also protect neurons from oxidative stress in their own capacity derived from inhibiting neurotoxic iron catalyzed by Fenton-based chemical reactions).

We also predict the presence of an active Iron-responsive Element in the ASN 5′UTR that accounts for the observed iron dependent translational control of α-synuclein (FIGS. 3 and 4). Like ferritin, α-synuclein expression appears to be controlled at the translational level by an active 5′ untranslated region Iron-responsive Element. We additionally directly test whether the α-synuclein 5′UTR binds to IRP-1 as the iron dependent mediator of ASN expression in neuronal cells in response to changed intracellular iron status, like the ferritin model.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references disclosed herein, including patent documents, are incorporated by reference in their entirety.

We claim:

1. An isolated nucleic acid molecule comprising: an α-synuclein translation enhancer element comprising the nucleotide sequence set forth as SEQ ID NO:1 or a fragment or variant thereof and a polypeptide-encoding nucleic acid sequence operably linked to the translation enhancer element.

2. The isolated nucleic acid molecule of claim 1, wherein the nucleotide sequence of the α-synuclein enhancer element comprises the sequence set forth as SEQ ID NO:1.

3. The isolated nucleic acid molecule of claim 1, wherein the fragment of SEQ ID NO:1 is SEQ ID NO:21.

4. The isolated nucleic acid molecule of claim 1, wherein the polypeptide-encoding nucleic acid sequence is a non-homologous polypeptide-encoding nucleic acid sequence.

5. The isolated nucleic acid of claim 1, wherein the 3′ nucleotide of the translation enhancer element is between 0 and about 100 nucleotides 5′ to the 5′ nucleotide of the polypeptide-encoding nucleic acid sequence.

6. (canceled)

7. A vector for expressing a recombinant polypeptide in a eukaryotic cell comprising:
a) a promoter that is active in the eukaryotic cell,
b) an α synuclein translation enhancer element comprising the nucleotide sequence set forth as SEQ ID NO:1 or a fragment or variant thereof, wherein the enhancer element is 3′ to the promoter; and
c) a nucleic acid sequence that encodes the recombinant polypeptide, wherein the nucleic acid sequence is 3′ to the translation enhancer element, and is operably linked to the promoter.

8. The vector of claim 7, wherein the nucleotide sequence of the α-synuclein translation enhancer element comprises the nucleotide sequence set forth as SEQ ID NO:1.

9. The vector of claim 7, wherein the fragment of SEQ ID NO:1 is SEQ ID NO:21.

10. The vector of claim 7, wherein the nucleic acid sequence is non-homologous to the translation-enhancer element.

11. The vector of claim 7, wherein the 3′ nucleotide of the translation enhancer element is between 0 and about 100 nucleotides 5′ to the 5′ nucleotide of the nucleic acid sequence.

12. (canceled)

13. An isolated host cell transformed with the vector of claim 7.

14. An isolated host cell transformed with the vector of claim 8.

15. A method for producing a recombinant polypeptide comprising:
a) growing host cells transformed with the vector of claim 7, and
b) purifying the recombinant polypeptide from either the host cells or the medium surrounding the host cells.

16. The method of claim 15, wherein the last 3′ nucleotide of the translation enhancer element is between 0 and about 100 nucleotides 5′ to the first 5′ nucleotide of the nucleic acid sequence.

17. (canceled)

18. (canceled)

19. The method of claim 15, further comprising contacting the transformed host cells with an inducer in an amount sufficient to significantly increase polypeptide production.

20-50. (canceled)


SEQUENCE LISTING (21 sequences)

SEQ ID NO: 1 - DNA, 52 bp, Homo sapiens
gctctcggagtggccattcgacgacagtgtggtgtaaaggaattcattagcc

SEQ ID NO: 2 - DNA, 3747 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaactccccactcctggtagctcagaccctctgatccaaccctccagccctgctgtgtgcccagagccaccttcctctcctaaacacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 3 - DNA, 1391 bp, Homo sapiens
ccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaaaaaaaaaaaa

SEQ ID NO: 4 - DNA, 2622 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 5 - DNA, 2529 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 6 - DNA, 52 bp, Homo sapiens
agtcgtcggggtttcctgcttcaacagtgcttggacggaacccggcgctcgt

SEQ ID NO: 7 - RNA, 24 nt, Homo sapiens
guggccauucgacgacaguguggu

SEQ ID NO: 8 - RNA, 24 nt, Homo sapiens
gguuuccugcuucaacagugcuug

SEQ ID NO: 9 - DNA, 21 bp, Artificial Sequence
  Synthetic Oligonucleotide
caccttctacaatgagctgcg

SEQ ID NO: 10 - DNA, 20 bp, Artificial Sequence
  Synthetic Oligonucleotide
gcacagcctggatagcaacg

SEQ ID NO: 11 - DNA, 21 bp, Artificial Sequence
  Synthetic Oligonucleotide
aggagccaggagaggacttcc

SEQ ID NO: 12 - DNA, 23 bp, Artificial Sequence
  Synthetic Oligonucleotide
tctccgacaactttctcttcagg

SEQ ID NO: 13 - DNA, 21 bp, Artificial Sequence
  Synthetic Oligonucleotide
tgacgggtgtgacagcagtag

SEQ ID NO: 14 - DNA, 17 bp, Artificial Sequence
  Synthetic Oligonucleotide
agccagtggctgctgca

SEQ ID NO: 15 - DNA, 21 bp, Artificial Sequence
  Synthetic Oligonucleotide
tcgactggactctgttcttga

SEQ ID NO: 16 - DNA, 21 bp, Artificial Sequence
  Synthetic Oligonucleotide
tggccaggtggaagtaaaatc

SEQ ID NO: 17 - RNA, 52 nt, Homo sapiens
gcucucggaguggccauucgacgacagugugguguaaaggaauucauuagcc

SEQ ID NO: 18 - RNA, 23 nt, Homo sapiens
cugcuucaacagugcuuggacgg

SEQ ID NO: 19 - RNA, 40 nt, Homo sapiens
  misc_feature (1)..(2): n = a, c, g, or u
nnggguuuccugcuucaacagugcuuggacggaacccnnn

SEQ ID NO: 20 - RNA, 27 nt, Homo sapiens
ccauucgacgacagugugguguaaagg

SEQ ID NO: 21 - DNA, 46 bp, Homo sapiens
ggagtggccattcgacgacagtgtggtgtaaaggaattcattagcc